(19) United States
(12) Patent Application Publication
(10) Pub. No.: US 2012/0258113 A1
(43) Pub. Date: Oct. 11, 2012

(54) SUSBSTANTIALLY PURE STEROIDOGENESIS INDUCING PEPTIDE AND USES THEREOF

(75) Inventor: Shafiq A. Khan, Mableton, GA (US)

(73) Assignee: CLARK ATLANTA UNIVERSITY, ATLANTA, GA (US)

(21) Appl. No.: 13/528,639

(22) Filed: Jun. 20, 2012

Related U.S. Application Data
(62) Division of application No. 11/799,994, filed on May 3, 2007, now abandoned.
(60) Provisional application No. 60/798,960, filed on May 9, 2006.

(51) Int. Cl. A61K 39/395; C12N 5/09; A61P 35/00

(52) U.S. Cl. 424/139.1; 435/375

(57) ABSTRACT

Provided are compositions and methods for increasing the production of steroid hormones in testes, ovaries, or adrenal glands in a subject in need thereof. Also provided are compositions and methods for inhibiting proliferation of ovarian, testicular, prostate gland, breast, and liver cancer cells.

### CROSS-REFERENCE TO RELATED APPLICATIONS

This application is a Divisional Application of U.S. patent application Ser. No. 11/799,994, filed on May 3, 2007, which claims benefit of U.S. Provisional Patent Application No. 60/798,960, filed on May 9, 2006, each of which is incorporated by reference herein in its entirety.

### ACKNOWLEDGMENTS

This invention was made with government support under Grant R03 HD043927 awarded by the National Institutes of Health. The government has certain rights in the invention.

### BACKGROUND OF THE INVENTION

1. Field of the Invention

This invention relates generally to a novel polypeptide that is involved with the production of various steroid hormones and with the proliferation of cancer cells. Specifically, the invention relates to a novel polypeptide that is involved in the production of adrenal, ovarian, and testicular hormones and which can be used to prepare antagonists that can be used to inhibit proliferation of cancer cells in an ovary, testis, prostate gland, breast, or liver.

2. Background Art

Steroidogenesis-inducing protein (SIP), a protein found in the ovaries of mammals, including humans, plays an important role in normal reproductive function in females, stimulates steroid production and proliferation of ovarian granulosa cells; these processes are essential for maturation of an ovarian follicle and ovulation. SIP is also produced in testes where it stimulates production of testosterone by testicular cells, and it is also a very potent mitogen for immature Leydig cells which develop in testes from precursor cells which proliferate and subsequently differentiate. If an insufficient number of Leydig cells develop in the testes of a subject, the testes will not produce enough testosterone for pubertal development and adult reproductive function. SIP is involved in the pre-pubertal increase in the number of Leydig cells in the testis resulting in adult levels of testosterone secretion which are required for normal male reproductive function. Thus, SIP plays a role in the secretion of testosterone by Leydig cells and a role in the proliferation and development of Leydig cells. SIP has been described in U.S. Pat. No. 5,484,767.

Ovarian cancer is a curable disease if it is detected early. However, due to the absence of simple screening procedures, the cancer is usually diagnosed in its later stages. The 5-year survival rate for patients diagnosed with ovarian cancer in the late stage of the disease is less than 30%. SIP stimulates proliferation of ovarian cancer cells, and cancer cells in advanced stages of the disease secrete SIP. Also, SIP can stimulate proliferation of testicular and prostate cancer cells. Therefore, SIP produced by cancer cells can provide a continuous stimulation for the proliferation of cancer cells and the growth of, for example, ovarian, testicular and prostate tumors. Furthermore, the presence of SIP in body fluids, for example blood, can provide a means to diagnose cancer, for example ovarian cancer, in early stages of the disease.

### SUMMARY OF THE INVENTION

Provided herein are methods and compositions for increasing production of a steroid hormone in a subject in need thereof. Also provided are methods and compositions for inhibiting proliferation of cancer cells in vitro and in vivo. Further provided is an antibody to a novel polypeptide that can detect SIP in a body fluid of a subject.

In accordance with the purposes of this invention, as embodied and broadly described herein, this invention, in one aspect, relates to a purified polypeptide fragment of a steroidogenesis inducing protein (SIP) having fewer amino acids than the entire steroidogenesis inducing protein, comprising an amino acid sequence identified as SEQ ID NO:2.

In another aspect, the invention relates to a method of increasing production of a steroid hormone in a subject in need thereof, comprising administering to the subject an effective amount of a purified polypeptide fragment of a steroidogenesis inducing protein having fewer amino acids than the entire steroidogenesis inducing protein, comprising an amino acid sequence identified as SEQ ID NO:2, and a pharmaceutically acceptable carrier. In yet another aspect, the invention relates to a method of increasing conversion of cholesterol to pregnenolone in a cell, comprising contacting the cell with a purified polypeptide fragment of a steroidogenesis inducing protein having fewer amino acids than the entire steroidogenesis inducing protein, comprising an amino acid sequence identified as SEQ ID NO:2, whereby contacting the cell increases conversion of cholesterol to pregnenolone in the cell.

In another aspect, the invention relates to a method of inhibiting the elaboration of steroidogenesis inducing protein (SIP) from a cell in a subject, comprising blocking the interaction of the cell with extracellular SIP, comprising contacting the extracellular SIP with an agent that binds to the extracellular SIP at a site of contact between the extracellular SIP and a cell membrane-bound receptor, thereby inhibiting interaction between the extracellular SIP with the receptor and inhibiting elaboration of SIP from the cell of the subject.

In another aspect, the invention relates to a method of inhibiting the proliferation of a cancer cell, comprising blocking the interaction of the cell with extracellular SIP, comprising contacting the extracellular SIP with an agent that binds to the extracellular SIP at a site of contact between the extracellular SIP and a cell membrane-bound receptor, thereby inhibiting interaction between the extracellular SIP with the receptor and inhibiting proliferation of the cell.

In another aspect, the invention relates to a method of screening for a compound that inhibits the interaction of extracellular SIP and a cell membrane-bound receptor of SIP, comprising the steps of: a) contacting the extracellular SIP with the compound; b) contacting a cell that expresses the cell membrane-bound receptor of SIP with the extracellular SIP; and c) determining whether the extracellular SIP of step a) is inhibited from binding to the membrane-bound receptor of SIP.

In another aspect, the invention relates to an isolated antibody that specifically binds a purified polypeptide fragment of a SIP having fewer amino acids than the entire SIP, comprising an amino acid sequence identified as SEQ ID NO:2.

In yet another aspect, the invention relates to a method of detecting SIP in a body fluid of a subject, comprising contacting the fluid with an antibody that specifically binds to a polypeptide fragment comprising an amino acid sequence identified as SEQ ID NO:2 under conditions wherein the antibody can bind to SIP, wherein the detection of SIP/antibody complexes indicates the presence of SIP in the body fluid of the subject.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1A shows SIP-specific antibodies, raised against synthetic polypeptide 2 consisting of the amino acid sequence identified as SEQ ID NO:2, recognize a 50 kD protein during several steps of purification (AS, S200, BS1, Hep1, PBE) and did not recognize a similar protein in fractions which did not contain SIP bioactivity (BS2, Hep2).

FIG. 1B shows neutralization of SIP effects on DNA synthesis in rat Leydig cells by SIP antibodies. Immature Leydig cells were cultured in the presence or absence of SIP antibodies (1:1000) and cells were treated with SIP. Antibodies completely blocked the stimulatory effects of SIP on DNA synthesis.

FIG. 2A shows a Western blot analysis that shows SIP is present in human ovarian follicular fluid (hFF), ascites fluid obtained from patients with advanced stages of ovarian cancer (AF), and in conditioned media from human granulosa cells (GC, hGLC, HGL5), porcine granulosa cells (pGL) and bovine luteal cells (bLC).

FIG. 2B shows a Western blot analysis of SIP in prostate cancer cells (PC3M, DU145), ovarian cancer cells (HEY) and liver cells (HepG2). S-200 represents partially purified SIP. These results indicate that SIP is produced both by normal ovarian cells and by prostate, ovarian and liver cancer cells. In addition, ovarian cancer patients produce this protein which accumulates in ascites fluid and provides a proliferatory signal for tumor cells.

FIG. 3A shows levels of SIP in serum and ascites fluid obtained from several patients with advanced ovarian cancer.

FIG. 3B shows the levels of SIP decrease in two patients after treatment.

FIG. 4A shows the effects of tyrosine kinase inhibitor, Genistein, on SIP effects of DNA synthesis in rat Leydig cells. The Leydig cells were cultured with SIP in the presence or absence of genistein, and effects were determined on proliferation. These results indicate that a tyrosine kinase coupled signaling pathway is required for this effect of SIP on target cells.

FIG. 4B shows the effects of tyrosine kinase inhibitor, Genistein, on SIP effects of steroidogenesis in rat Leydig cells. The Leydig cells were cultured with SIP in the presence or absence of genistein, and effects were determined on steroid production. These results indicate that a tyrosine kinase coupled signaling pathway is required for this effect of SIP on target cells.

FIGS. 5A-C show activation of various components of MAP-kinase signaling pathway in Leydig cells by SIP. Leydig cells were treated with SIP or EGF (used as a positive control) for 30 minutes. The activation of Ras, MEK and ERK proteins were determined using activation specific immunoblots. Actin western blots were used as protein loading controls.

FIG. 6 shows activation of PI3-kinase/AKT pathway by SIP in rat Leydig cells. Leydig cells were treated with SIP or EGF for 30 min, and the activation of AKT and its downstream target (p70 S6K) were determined using activation specific antibodies. EGF was used as a positive control, and Actin blots were run as loading controls. The data in FIGS. 6 and 7 show that like many growth factors, tyrosine kinase coupled signaling pathway leads to activation of both MAP-kinase and AKT pathways. These pathways are required for growth factor effects on survival and proliferation of target cells.

FIG. 7A shows that inhibition of MAP-kinase (PD98059) and AKT (LY294002) by specific inhibitors blocks the effects of SIP on DNA synthesis in Leydig cells but not on steroid production, FIG. 7B. These results suggest that SIP effects on proliferation require both of these pathways. The steroidogenic effect of SIP appears to be mediated by an alternative intracellular pathway.

FIG. 8 shows that stimulation of DNA synthesis in rat Leydig cells by purified SIP (S200 and HEP) and by peptide 2 ((P) (SEQ ID NO:2)). LH and EGF were used as positive controls. These data show that, like whole SIP protein, peptide 2 is capable of inducing proliferation of immature rat Leydig cells.

FIG. 9 shows stimulation of DNA synthesis by purified SIP (S200 and HEP) and different doses of synthetic SIP peptide 2 (P) on DNA synthesis in human ovarian epithelial cancer (HEY) cells. FBS was used as a positive control. These results suggest that synthetic SIP 2 peptide (SEQ ID NO:2) is capable of exerting mitogenic effects in human ovarian epithelial cancer cells similar to those shown to full length purified SIP.

FIG. 10 shows the effects of SIP and synthetic peptide 2 on DNA synthesis in human prostate cancer (PC3M) cells.

FIG. 11 shows stimulation of testosterone production in rat Leydig cells by purified SIP and synthetic SIP peptide 2 (SEQ ID NO:2). The cells were cultured in the presence of LH (positive control), SIP, or synthetic peptide 2 for 6, 24 or 48 hours. Testosterone produced by Leydig cells was determined using radioimmunoassay. These data show that synthetic peptide 2 retains the steroidogenic activity of the full length protein, SIP, although the effects of peptide 2 are not as high as those observed with purified SIP. * represents statistically significant compared to untreated control cells.

FIG. 12A shows SIP and synthetic peptide 2 (P, used at different doses) induce the expression of StAR mRNA. FIG. 12B shows SIP and synthetic peptide 2 (P, used at different doses) induce the expression of protein in rat Leydig cells. Leydig cells were treated for 24 hour and StAR mRNA and protein were determined using RT-PCR and Western blotting, respectively. StAR protein is required for transport of cholesterol into the mitochondria where it is converted to pregnenolone and which is a rate-limiting step in all steroidogenic cells. LH was used as a positive control.

FIG. 13 shows effects of SIP and synthetic peptide 2 (P, used as different doses) on tyrosine phosphorylation of Leydig cell proteins. Leydig cells were treated for 30 minutes, and total proteins were analyzed for tyrosine phosphorylation using phopho-tyrosine specific antibodies. EGF was used as positive control. The data show that, like purified SIP, synthetic peptide 2 is capable of binding to SIP receptors and initiating signaling cascade required for SIP effects on target cells.

FIG. 14 shows effects of purified SIP and synthetic SIP peptide 2 on activation of MAP-kinase and AKT pathway in Leydig cells. These data show that both pathways are activated by peptide 2 (SEQ ID NO:2) similar to the full length purified SIP. LH and EGF were used as controls.

### DESCRIPTION OF THE PREFERRED EMBODIMENTS

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific synthetic methods, specific polypeptides, or to particular nucleic acids, as such may, of course, vary. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Thus, for example, reference to “a purified polypeptide” includes mixtures of two or more polypeptides; reference to “a pharmaceutical carrier” includes mixtures of two or more such carriers, and the like.

Ranges may be expressed herein as from “about” one particular value, and/or to “about” another particular value. When such a range is expressed, another embodiment includes, from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

In this specification and in the claims which follow, reference will be made to a number of terms which shall be defined to have the following meanings: “Optional” or “optionally” means that the subsequently described event or circumstance may or may not occur, and that the description includes instances where said event or circumstance occurs and instances where it does not. For example, the phrase “the polypeptide can optionally be in a pharmaceutically acceptable carrier” means that the polypeptide may or may not be in a pharmaceutically acceptable carrier.

Provided herein is a purified polypeptide fragment of a steroidogenesis inducing protein (SIP) having fewer amino acids than the entire steroidogenesis inducing protein, comprising an amino acid sequence identified as SEQ ID NO:2. As used herein, “peptide 2” is a novel purified polypeptide consisting of an amino acid sequence identified as SEQ ID NO:2. As used herein, a “steroidogenesis inducing protein” is a protein that induces, stimulates, or increases the production of a steroid hormone in a cell of a subject.

As used herein, “a steroid hormone” includes, but is not limited to, a hormone produced in the ovaries, testes, and adrenal glands of a subject. Examples of hormones produced in an ovary of a subject include, but are not limited to, testosterone, estrogen, and progesterone. In an ovary, testosterone is converted to estrogen by aromatase, an enzyme in granulosa cells. An example of a hormone produced in a testis of a subject is testosterone. Examples of hormones produced in an adrenal gland of a subject include, but are not limited to, glucocorticoids (e.g., cortisol, or hydrocortisone), mineralocorticoids (e.g., aldosterone), and androgens (e.g., testosterone). As used herein, by “subject” is meant and individual. Preferably, the subject is a mammal such as a primate, and more preferably a human. The term “subject” includes domesticated animals such as cats, dogs, etc., livestock (e.g., cattle, horses, pigs, sheep, goats, etc.), and laboratory animals (e.g., mice, rabbits, rats, gerbils, guinea pigs, etc.).

Steroidogenesis is regulated at two different levels. Chronic and long term regulation involves the maintenance of all steroidogenic enzymes in a cell which are required to synthesize tissue-specific steroids. Acute regulation involves an increase in steroid biosynthesis in response to hormonal stimulation. This is a very quick process that can change levels of steroids within minutes. This acute increase in steroid production is dependent on one rate-limiting step in all steroidogenic tissues such as testis, ovary, and adrenal gland. The enzyme P450scc, which converts cholesterol to pregnenolone, is localized in the inner mitochondrial membrane. However, cholesterol can not get to the inner mitochondrial membrane because it is insoluble in water and, hence, can not cross the space between outer and inner mitochondrial membranes. Hormones, for example hCG, LH, ACTH and SIP, act at this step by increasing the amount of steroidogenic acute regulatory protein (StAR) which transports cholesterol into the mitochondria. Because this step is common in all steroidogenic tissues, SIP can stimulate conversion of cholesterol to pregnenolone in testicular, ovarian and adrenal cells. Pregnenolone then moves back to the cytoplasm and is converted into other steroids which are specific for different tissues and depend upon the presence of specific enzymes which convert pregnenolone into other products. In testis, the target cells produce testosterone. If SIP or a synthetic peptide that has the activity of SIP is delivered locally to a specific tissue, it can stimulate the production of steroids which are specific to that tissue. For example, SIP or a synthetic peptide, for example peptide 2 (SEQ ID NO:2), that has the activity of SIP causes testosterone production in testis, progesterone, testosterone and estrogen production in ovarian cells, and corticosteroid, mineralocorticoid, and androgen production in adrenal cells.

In one aspect, the purified polypeptide fragment of SIP has an amino acid sequence greater than about 95% similar to an amino acid sequence identified as SEQ ID NO:2. Thus, the purified polypeptide fragment can be greater than about 95%, 96%, 97%, 98%, or 99% similar to an amino acid sequence identified as SEQ ID NO:2. In another aspect, the purified polypeptide fragment of SIP consists essentially of an amino acid sequence identified as SEQ ID NO:2. In another aspect, the purified polypeptide fragment of SIP consists of an amino acid sequence (DVNGGGATLPQPLYQTA) identified as SEQ ID NO:2.

As used herein, a “purified polypeptide” or a “purified polypeptide fragment” is a polypeptide, or a fragment of a polypeptide, that is substantially free from the materials with which the polypeptide, or a fragment of a polypeptide, is normally associated in nature or in culture. The polypeptides of the invention can be obtained, for example, by extraction from a natural source, for example, an isolated cell from, for example, a testis, an ovary, or an adrenal gland; by expression of a recombinant nucleic acid encoding the polypeptide (for example, in a cell or in a cell-free translation system); or by chemically synthesizing the polypeptide. In addition, a polypeptide may be obtained by cleaving full-length polypeptides. When the polypeptide is a fragment of a larger naturally occurring polypeptide, the purified (isolated) polypeptide is shorter than and excludes the full-length, naturally-occurring polypeptide of which it is a fragment. For example, the purified polypeptide fragment comprising the amino acid sequence identified as SEQ ID NO:2 is a novel purified fragment of SIP, and, like SIP, can induce steroidogenesis in a cell of a subject in need thereof.

The disclosed purified polypeptide fragments of SIP can be prepared by using any of a number of chemical polypeptide synthesis techniques well-known to those of ordinary skill in the art, including solution methods and solid phase methods. One method of producing the disclosed polypeptides is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to an antigen of the present invention, for example, can be synthesized by standard chemical reactions.

For example, enzymatic ligation of cloned or synthetic peptide segments allows relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two-step chemical reaction (Dawson et al. Synthesis of Proteins by Native Chemical Ligation. Science, 266:776-779 (1994)). The first step is the chemoselective reaction of an unprotected synthetic peptide-alpha-thioester with another unprotected peptide segment containing an amino-terminal Cys residue to give a thioester-linked intermediate as the initial covalent product. Without a change in the reaction conditions, this intermediate undergoes spontaneous, rapid intra-molecular reaction to form a native peptide bond at the ligation site. Application of this native chemical ligation method to the total synthesis of a protein molecule is illustrated by the preparation of human interleukin 8 (IL-8) (Baggiolini M et al. (1992) FEBS Lett. 307:97-101; Clark-Lewis I et al., J. Biol. Chem., 269:16075 (1994); Clark-Lewis 1 et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

The polypeptides of the invention can also be prepared by other means including, for example, recombinant techniques. Examples of appropriate cloning and sequencing techniques, and instructions sufficient to direct persons of skill through many cloning exercises are found in Sambrook et al. (2001) Molecular Cloning A Laboratory Manual (3rd ed.) Vol. 1-3, Cold Spring Harbor Laboratory, Cold Spring Harbor Press, NY, (Sambrook).

It is understood that as discussed herein, the terms “similar” or “similarity” mean the same thing as “homology” and “identity.” Thus, for example, if the use of the word homology is used to refer to two non-natural sequences, it is understood that this is not necessarily indicating an evolutionary relationship between these two sequences, but rather is looking at the similarity or relatedness between their nucleic acid or amino acid sequences. Many of the methods for determining similarity between two evolutionarily related molecules are routinely applied to any two or more nucleic acids or polypeptides for the purpose of measuring sequence similarity regardless of whether they are evolutionarily related.

In general, it is understood that one way to define any known variants and derivatives or those that might arise, of the disclosed nucleic acids and polypeptides herein, is through defining the variants and derivatives in terms of similarity, or homology, to specific known sequences. In general, variants of nucleic acids and polypeptides herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent similarity, or homology, to the stated sequence or the native sequence. Those of skill in the art readily understand how to determine the similarity of two polypeptides or nucleic acids. For example, the similarity can be calculated after aligning the two sequences so that the similarity is at its highest level.

Another way of calculating similarity, or homology, can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.; the BLAST algorithm of Tatusova and Madden FEMS Microbiol. Lett. 174: 247-250 (1999) available from the National Center for Biotechnology Information (http://www.ncbi.nlm nih gov/blast/bl2seq/bl2.html)), or by inspection.

The same types of similarity, or homology, can be obtained for nucleic acids by, for example, the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if similarity is found with at least one of these methods, the sequences would be said to have the stated similarity.

For example, as used herein, a sequence recited as having a particular percent similarity to another sequence refers to sequences that have the recited similarity as calculated by any one or more of the calculation methods described above. For example, a first sequence has 80 percent similarity, as defined herein, to a second sequence if the first sequence is calculated to have 80 percent similarity to the second sequence using the Zuker calculation method even if the first sequence does not have 80 percent similarity to the second sequence as calculated by any of the other calculation methods. As another example, a first sequence has 80 percent similarity, as defined herein, to a second sequence if the first sequence is calculated to have 80 percent similarity to the second sequence using both the Zuker calculation method and the Pearson and Lipman calculation method even if the first sequence does not have 80 percent similarity to the second sequence as calculated by the Smith and Waterman calculation method, the Needleman and Wunsch calculation method, the Jaeger calculation methods, or any of the other calculation methods. As yet another example, a first sequence has 80 percent similarity, as defined herein, to a second sequence if the first sequence is calculated to have 80 percent similarity to the second sequence using each of the calculation methods (although, in practice, the different calculation methods will often result in different calculated similarity percentages).

Each of the disclosed polypeptides can have one or more conservative amino acid substitutions. These conservative substitutions are such that a naturally occurring amino acid is replaced by one having similar properties. Such conservative substitutions do not alter the function of the polypeptide. For example, conservative substitutions can be made according to Table 1, shown below.

Thus, it is understood that, where desired, modifications and changes may be made in the nucleic acid encoding the polypeptides of this invention and/or in the amino acid sequence of the disclosed polypeptides and still obtain a polypeptide having like or otherwise desirable characteristics. Such changes may occur in natural isolates or may be synthetically introduced using site-specific mutagenesis, the procedures for which, such as mis-match polymerase chain reaction (PCR), are well known in the art. For example, certain amino acids may be substituted for other amino acids in a polypeptide without appreciable loss of functional activity. It is thus contemplated that various changes may be made in the amino acid sequence of the polypeptides of the present invention (or underlying nucleic acid sequence) without appreciable loss of biological utility or activity and possibly with an increase in such utility or activity.

The disclosed purified polypeptides can be used, for example, to induce, stimulate, and/or increase production of a steroid hormone in a cell of a subject in a tissue-specific manner. Because the disclosed polypeptide fragments are smaller than the full-length SIP, the fragments can be administered more efficiently to a subject in need thereof.

Provided herein is an isolated nucleic acid encoding a polypeptide comprising an amino acid sequence identified as SEQ ID NO:2. Also provided is an isolated nucleic acid encoding a polypeptide consisting of an amino acid sequence identified as SEQ ID NO:2. An example of a nucleic acid that encodes the polypeptide consisting of an amino acid sequence identified as SEQ ID NO:2 is the nucleic acid (gat gtg aac ggc ggc ggc gcg acc ctg ccg cag ccg ctg tat cag acc gca), identified as SEQ ID NO:4. Other nucleic acid sequences include, but are not limited to, GAY ATY AAY GGN GGN GGN GCN ACN NNN CCN CAR CCN NNN NNN CAR ACN GCN where N=any base; Y=C or T; R=A or G.

As used herein, the term “nucleic acid” refers to single or multiple stranded molecules which may be DNA or RNA, or any combination thereof, including modifications to those nucleic acids. The nucleic acid may represent a coding strand or its complement, or any combination thereof. Nucleic acids may be identical in sequence to the sequences which are naturally occurring for any of the moieties discussed herein or may include alternative codons which encode the same amino acid as that which is found in the naturally occurring sequence. These nucleic acids can also be modified from their typical structure. Such modifications include, but are not limited to, methylated nucleic acids, the substitution of a non-bridging oxygen on the phosphate residue with either a sulfur (yielding phosphorothioate deoxynucleotides), selenium (yielding phosphorselenoate deoxynucleotides), or methyl groups (yielding methylphosphonate deoxynucleotides), a reduction in the AT content of AT rich regions, or replacement of non-preferred codon usage of the expression system to preferred codon usage of the expression system. The nucleic acid can be directly cloned into an appropriate vector, or if desired, can be modified to facilitate the subsequent cloning steps. Such modification steps are routine, an example of which is the addition of oligonucleotide linkers which contain restriction sites to the termini of the nucleic acid. General methods are set forth in in Sambrook et al. (2001) Molecular Cloning—A Laboratory Manual (3rd ed.) Vol. 1-3, Cold Spring Harbor Laboratory, Cold Spring Harbor Press, NY, (Sambrook).

The nucleic acids of this invention can be detected with a probe capable of hybridizing to the nucleic acid of a cell or a sample. This probe can be a nucleic acid comprising the nucleotide sequence of a coding strand or its complementary strand or the nucleotide sequence of a sense strand or antisense strand, or a fragment thereof. Thus, a probe of this invention can be either DNA or RNA and can bind either DNA or RNA, or both, in the biological sample. The nucleic acids of the present invention or any of the other disclosed nucleic acids, and fragments thereof, can be utilized as probes or primers to detect other nucleic acids that encode SIP. A polynucleotide probe or primer comprising at least 15 contiguous nucleotides can be utilized. Therefore, the polynucleotide probes or primers of this invention can be at least 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195 or at least 200 nucleotides in length.

As used herein, the term “nucleic acid probe” refers to a nucleic acid fragment that selectively hybridizes under stringent conditions with a nucleic acid comprising a nucleic acid set forth in a sequence listed herein. This hybridization must be specific. The degree of complementarity between the hybridizing nucleic acid and the sequence to which it hybridizes should be at least enough to exclude hybridization with a nucleic acid encoding an unrelated protein.

“Stringent conditions” refers to the washing conditions used in a hybridization protocol. In general, the washing conditions should be a combination of temperature and salt concentration chosen so that the denaturation temperature is approximately 5° C. to 20° C. below the calculated Tm of the nucleic acid hybrid under study. The temperature and salt conditions are readily determined empirically in preliminary experiments in which samples of reference DNA immobilized on filters are hybridized to the probe or polypeptide-coding nucleic acid of interest and then washed under conditions of different stringencies. The Tm of such an oligonucleotide can be estimated by allowing 2° C. for each A or T nucleotide, and 4° C. for each G or C. For example, an 18 nucleotide probe of 50% G+C would, therefore, have an approximate Tm of 54° C. Stringent conditions are known to one of skill in the art. See, for example, Sambrook et al. (2001). The following is an exemplary set of hybridization conditions and is not limiting:

### Very High Stringency

Hybridization: 5×SSC at 65° C. for 16 hours
Wash twice: 2×SSC at room temperature (RT) for 15 minutes each
Wash twice: 0.5×SSC at 65° C. for 20 minutes each

### High Stringency

Hybridization: 5×-6×SSC at 65° C.-70° C. for 16-20 hours
Wash twice: 2×SSC at RT for 5-20 minutes each
Wash twice: 1×SSC at 55° C.-70° C. for 30 minutes each

### Low Stringency

Hybridization: 6×SSC at RT to 55° C. for 16-20 hours
Wash at least twice: 2×-3×SSC at RT to 55° C. for 20-30 minutes each.

As mentioned above, the disclosed nucleic acids and fragments thereof can be utilized as primers to amplify a disclosed nucleic acid by standard amplification techniques. A variety of PCR techniques are familiar to those skilled in the art. For a review of PCR technology, see White (1997) and the publication entitled “PCR Methods and Applications” (1991, Cold Spring Harbor Laboratory Press), which is incorporated herein by reference in its entirety for amplification methods. In each of these PCR procedures, PCR primers on either side of the nucleic acid sequences to be amplified are added to a suitably prepared nucleic acid sample along with dNTPs and a thermostable polymerase such as Taq polymerase, Pfu polymerase, or Vent polymerase. The nucleic acid in the sample is denatured and the PCR primers are specifically hybridized to complementary nucleic acid sequences in the sample. The hybridized primers are extended. Thereafter, another cycle of denaturation, hybridization, and extension is initiated. The cycles are repeated multiple times to produce an amplified fragment containing the nucleic acid sequence between the primer sites. PCR has further been described in several patents including U.S. Pat. Nos. 4,683,195, 4,683,202 and 4,965,188. Each of these publications is incorporated herein by reference in its entirety for PCR methods. One of skill in the art would know how to design and synthesize primers that amplify the disclosed nucleic acids or fragments thereof.

A detectable label may be included in an amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g., ³²P, ³⁵S, ³H, etc. The label may be a two stage system, where the amplified DNA is conjugated to biotin, haptens, etc., having a high affinity binding partner, e.g., avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acid, for example amplified fragment, can be analyzed by one of a number of methods known in the art. The nucleic acid can be sequenced by dideoxy or other methods. Hybridization with the sequence can also be used to determine its presence, by Southern blots, dot blots, etc.

Once the nucleic acid sequence is obtained, the sequence encoding the specific amino acids can be modified or changed at any particular amino acid position by techniques well known in the art. For example, PCR primers can be designed which span the amino acid position or positions and which can substitute any amino acid for another amino acid. Alternatively, one skilled in the art can introduce specific mutations at any point in a particular nucleic acid sequence through techniques for point mutagenesis. General methods are set forth in Smith, M. “In vitro mutagenesis” Ann Rev. Gen., 19:423-462 (1985) and Zoller, M. J. “New molecular biology methods for protein engineering” Cum Opin. Struct. Biol., 1:605-610 (1991), which are incorporated herein in their entirety for the methods. These techniques can be used to alter the coding sequence without altering the amino acid sequence that is encoded.

In another aspect, provided is a vector, comprising a disclosed nucleic acid. An example of a disclosed nucleic acid includes, but is not limited to, the nucleic acid identified as SEQ ID NO:4. The vector can direct the in vivo or in vitro synthesis of any of the polypeptides described herein. The vector is contemplated to have the necessary functional elements that direct and regulate transcription of the inserted nucleic acid. These functional elements include, but are not limited to, a promoter, regions upstream or downstream of the promoter, such as enhancers that may regulate the transcriptional activity of the promoter, an origin of replication, appropriate restriction sites to facilitate cloning of inserts adjacent to the promoter, antibiotic resistance genes or other markers which can serve to select for cells containing the vector or the vector containing the insert, RNA splice junctions, a transcription termination region, or any other region which may serve to facilitate the expression of the inserted gene or hybrid gene. (See generally, Sambrook et al.). The vector, for example, can be a plasmid. The vectors can contain genes conferring hygromycin resistance, gentamicin resistance, or other genes or phenotypes suitable for use as selectable markers, or methotrexate resistance for gene amplification. The vector can comprise the nucleic acid in pET15b, pSRα-Neo, pPICZα, or pPIC9K.

There are numerous E. coli (Escherichia coli) expression vectors, known to one of ordinary skill in the art, which are useful for the expression of the nucleic acid insert. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which will typically contain expression control sequences compatible with the host cell (e.g., an origin of replication). In addition, any number of a variety of well-known promoters will be present, such as the lactose promoter system, a tryptophan (Trp) promoter system, a beta-lactamase promoter system, or a promoter system from phage lambda. The promoters will typically control expression, optionally with an operator sequence, and have ribosome binding site sequences, for example, for initiating and completing transcription and translation. If necessary, an amino terminal methionine can be provided by insertion of a Met codon 5′ and in-frame with the downstream nucleic acid insert. Also, the carboxy-terminal extension of the nucleic acid insert can be removed using standard oligonucleotide mutagenesis procedures. Also, nucleic acid modifications can be made to promote amino terminal homogeneity.

Additionally, yeast expression can be used. The invention provides a nucleic acid encoding a polypeptide of the present invention, wherein the nucleic acid can be expressed by a yeast cell. More specifically, the nucleic acid can be expressed by Pichia pastoris or S. cerevisiae. There are several advantages to yeast expression systems, which include, for example, Saccharomyces cerevisiae and Pichia pastoris. First, evidence exists that proteins produced in a yeast secretion systems exhibit correct disulfide pairing. Second, efficient large scale production can be carried out using yeast expression systems. The Saccharomyces cerevisiae pre-pro-alpha mating factor leader region (encoded by the MFa-1 gene) can be used to direct protein secretion from yeast (Brake, et al.). The leader region of pre-pro-alpha mating factor contains a signal peptide and a pro-segment which includes a recognition sequence for a yeast protease encoded by the KEX2 gene: this enzyme cleaves the precursor protein on the carboxyl side of a Lys-Arg dipeptide cleavage signal sequence. The nucleic acid coding sequence can be fused in-frame to the pre-pro-alpha mating factor leader region. This construct can be put under the control of a strong transcription promoter, such as the alcohol dehydrogenase I promoter, alcohol oxidase I promoter, a glycolytic promoter, or a promoter for the galactose utilization pathway. The nucleic acid coding sequence is followed by a translation termination codon which is followed by transcription termination signals. Alternatively, the nucleic acid coding sequences can be fused to a second protein coding sequence, such as Sj26 or beta-galactosidase, used to facilitate purification of the fusion protein by affinity chromatography. The insertion of protease cleavage sites to separate the components of the fusion protein is applicable to constructs used for expression in yeast. Efficient post translational glycosylation and expression of recombinant proteins can also be achieved in Baculovirus systems.

The invention also provides for the vectors containing the contemplated nucleic acids in a host suitable for expressing the nucleic acids. The host cell can be a prokaryotic cell, including, for example, a bacterial cell. More particularly, the bacterial cell can be an E. coli cell. Alternatively, the cell can be a eukaryotic cell, including, for example, a Chinese hamster ovary (CHO) cell, a myeloma cell, a Pichia cell, or an insect cell. The coding sequence for any of the polypeptides described herein can be introduced into a Chinese hamster ovary (CHO) cell line, for example, using a methotrexate resistance-encoding vector, or other cell lines using suitable selection markers. Presence of the vector DNA in transformed cells can be confirmed by Southern blot analysis. Production of RNA corresponding to the insert coding sequence can be confirmed by Northern blot analysis. A number of other suitable host cell lines have been developed and include myeloma cell lines, fibroblast cell lines, and a variety of tumor cell lines such as melanoma cell lines. Expression vectors for these cells can include expression control sequences, such as an origin of replication, a promoter, an enhancer, and necessary information processing sites, such as ribosome binding sites, RNA splice sites, polyadenylation sites, and transcriptional terminator sequences. Preferred expression control sequences are promoters derived from immunoglobulin genes, SV40, Adenovirus, Bovine Papilloma Virus, etc. The vectors containing the nucleic acid segments of interest can be transferred into the host cell by well-known methods, which vary depending on the type of cellular host. For example, calcium chloride transformation is commonly utilized for prokaryotic cells, whereas calcium phosphate, DEAE dextran, or lipofectin mediated transfection or electroporation may be used for other cellular hosts.

Mammalian cells permit the expression of proteins in an environment that favors important post-translational modifications such as folding and cysteine pairing, addition of complex carbohydrate structures, and secretion of active protein. Vectors useful for the expression of active proteins in mammalian cells are characterized by insertion of the protein coding sequence between a strong viral promoter and a polyadenylation signal. The vectors can contain genes conferring hygromycin resistance, genticin or G418 resistance, or other genes or phenotypes suitable for use as selectable markers, or methotrexate resistance for gene amplification. The chimeric protein coding sequence can be introduced into a Chinese hamster ovary (CHO) cell line using a methotrexate resistance-encoding vector, or other cell lines using suitable selection markers. Presence of the vector DNA in transformed cells can be confirmed by Southern blot analysis. Production of RNA corresponding to the insert coding sequence can be confirmed by Northern blot analysis. A number of other suitable host cell lines capable of secreting intact human proteins have been developed in the art, and include the CHO cell lines, HeLa cells, myeloma cell lines, Jurkat cells, etc. Expression vectors for these cells can include expression control sequences, such as an origin of replication, a promoter, an enhancer, and necessary information processing sites, such as ribosome binding sites, RNA splice sites, polyadenylation sites, and transcriptional terminator sequences. Preferred expression control sequences are promoters derived from immunoglobulin genes, SV40, Adenovirus, Bovine Papilloma Virus, etc. The vectors containing the nucleic acid segments of interest can be transferred into the host cell by well-known methods, which vary depending on the type of cellular host. For example, calcium chloride transformation is commonly utilized for prokaryotic cells, whereas calcium phosphate, DEAE dextran, or lipofectin mediated transfection or electroporation may be used for other eukaryotic cellular hosts.

Alternative vectors for the expression of genes or nucleic acids in mammalian cells, those similar to those developed for the expression of human gamma-interferon, tissue plasminogen activator, clotting Factor VIII, hepatitis B virus surface antigen, protease Nexin1, and eosinophil major basic protein, can be employed. Further, the vector can include CMV promoter sequences and a polyadenylation signal available for expression of inserted nucleic acids in mammalian cells (such as COS-7).

Insect cells also permit the expression of mammalian proteins. Recombinant proteins produced in insect cells with baculovirus vectors undergo post-translational modifications similar to that of wild-type proteins. Briefly, baculovirus vectors useful for the expression of active proteins in insect cells are characterized by insertion of the protein coding sequence downstream of the Autographica californica nuclear polyhedrosis virus (AcNPV) promoter for the gene encoding polyhedrin, the major occlusion protein. Cultured insect cells such as Spodoptera frugiperda cell lines are transfected with a mixture of viral and plasmid DNAs and the viral progeny are plated. Deletion or insertional inactivation of the polyhedrin gene results in the production of occlusion negative viruses which form plaques that are distinctively different from those of wild-type occlusion positive viruses. These distinctive plaque morphologies allow visual screening for recombinant viruses in which the AcNPV gene has been replaced with a hybrid gene of choice.

Thus provided is a method of making any of the disclosed purified polypeptides, fragments and variants described herein, comprising culturing a host cell comprising a vector that encodes a SIP polypeptide, fragment or variant thereof, and purifying the polypeptide produced by the host cell. As mentioned above, these polypeptides include, but are not limited to, a purified polypeptide fragment of SIP having fewer amino acids than the entire SIP, comprising an amino acid sequence identified as SEQ ID NO:2, polypeptides comprising an amino acid sequence greater than about 95% similar to the sequence of SEQ ID NO:2 or a fragment thereof, polypeptides comprising the amino acid sequence of SEQ ID NO:2, or a fragment thereof, with one or more conservative amino acid substitutions, polypeptides consisting essentially of the amino acid sequence identified as SEQ ID NO:2, and polypeptides consisting of an amino acid sequence identified as SEQ ID NO:2.

Provided is a composition comprising an isolated nucleic acid encoding a disclosed purified polypeptide fragment of SIP having fewer amino acids than the entire SIP, comprising an amino acid sequence identified as SEQ ID NO:2 and a pharmaceutically acceptable carrier. For example, provided is an isolated nucleic acid encoding a purified polypeptide fragment of SIP having an amino acid sequence greater than about 95% similar to an amino acid sequence identified as SEQ ID NO:2 and a pharmaceutically acceptable carrier. In another aspect, provided is an isolated nucleic acid encoding a purified polypeptide fragment of SIP consisting essentially of an amino acid sequence identified as SEQ ID NO:2 and a pharmaceutically acceptable carrier. In yet another aspect, provided is an isolated nucleic acid encoding a purified polypeptide fragment of SIP consisting of an amino acid sequence identified as SEQ ID NO:2 and a pharmaceutically acceptable carrier.

Further, provided is a composition comprising a vector comprising an isolated nucleic acid encoding a purified polypeptide fragment of SIP and a pharmaceutically acceptable carrier. For example, provided is a composition comprising a vector comprising an isolated nucleic acid encoding a purified polypeptide fragment of SIP having fewer amino acids than the entire SIP, comprising an amino acid sequence identified as SEQ ID NO:2 and a pharmaceutically acceptable carrier. In another aspect, the vector can comprise an isolated nucleic acid encoding a purified polypeptide fragment of SIP having an amino acid sequence greater than about 95% similar to an amino acid sequence identified as SEQ ID NO:2 and a pharmaceutically acceptable carrier. In another aspect, provided is a vector comprising an isolated nucleic acid encoding a purified polypeptide fragment of SIP consisting essentially of an amino acid sequence identified as SEQ ID NO:2 and a pharmaceutically acceptable carrier. In yet another aspect, provided is a vector comprising an isolated nucleic acid encoding a purified polypeptide fragment of SIP consisting of an amino acid sequence identified as SEQ ID NO:2 and a pharmaceutically acceptable carrier.

Further provided is a composition comprising the disclosed polypeptides, fragments and variants thereof, and a pharmaceutically acceptable carrier. For example, provided is a composition comprising a purified polypeptide fragment of SIP having fewer amino acids than the entire SIP, comprising an amino acid sequence identified as SEQ ID NO:2 and a pharmaceutically acceptable carrier. In one aspect, the composition comprises a purified polypeptide having greater than about 95% similarity to an amino acid sequence identified as SEQ ID NO:2 and a pharmaceutically acceptable carrier. In another aspect, the composition comprises a purified polypeptide consisting essentially of an amino acid sequence identified as SEQ ID NO:2 and a pharmaceutically acceptable carrier. In yet another aspect, the composition comprises a purified polypeptide consisting of an amino acid sequence identified as SEQ ID NO:2 and a pharmaceutically acceptable carrier. By “pharmaceutically acceptable carrier” is meant a material that is not biologically or otherwise undesirable, i.e., the material may be administered to a subject, along with the substance, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Pharmaceutical carriers are known to those skilled in the art. These most typically would be standard carriers for administration of drugs to humans, including solutions such as sterile water, saline, and buffered solutions at physiological pH. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Provided is an isolated (purified) antibody or fragment thereof, that specifically binds SIP or a purified polypeptide fragment of SIP having fewer amino acids than the entire SIP, comprising an amino acid sequence identified as SEQ ID NO:2. As used herein, an “isolated (purified) antibody or fragment thereof” is an antibody, or a fragment of an antibody, that is substantially free from the materials with which the antibody, or a fragment of an antibody, is normally associated in nature or in culture. In one aspect, provided is an isolated antibody that specifically binds a purified polypeptide fragment of SIP that has an amino acid sequence greater than about 95% similar to an amino acid sequence identified as SEQ ID NO:2. In another aspect, provided is an isolated antibody that specifically binds a purified polypeptide fragment of SIP consisting essentially of an amino acid sequence identified as SEQ ID NO:2. In another aspect, provided is an isolated antibody that specifically binds a purified polypeptide fragment of SIP consisting of an amino acid sequence identified as SEQ ID NO:2.

The antibody of the present invention can be a polyclonal antibody or a monoclonal antibody. The antibody of the invention selectively binds a SIP polypeptide. By “selectively binds” or “specifically binds” is meant an antibody binding reaction which is determinative of the presence of the antigen (in the present case, a SIP polypeptide) or antigenic fragments thereof among a heterogeneous population of proteins and other biologics. Thus, under designated immunoassay conditions, the specified antibodies bind preferentially to a particular peptide and do not bind in a significant amount to other proteins in the sample. Specific binding to a SIP polypeptide under such conditions requires an antibody that is selected for its specificity to a SIP polypeptide. Preferably, selective binding includes binding at about or above 1.5 times assay background and the absence of significant binding is less than 1.5 times assay background.

This invention also contemplates antibodies that compete for binding to natural SIP interactors. For example, an antibody of the present invention can compete with SIP for a binding site (e.g. a receptor) on a cell or the antibody can compete with SIP for binding to another protein or biological molecule. The antibody optionally can have either an antagonistic or agonistic function as compared to the antigen.

Preferably, the antibody binds a SIP polypeptide ex vivo or in vivo. Optionally, the antibody of the invention is labeled with a detectable moiety. For example, the detectable moiety can be selected from the group consisting of a fluorescent moiety, an enzyme-linked moiety, a biotin moiety and a radiolabeled moiety. The antibody can be used in techniques or procedures such as diagnostics, therapeutics, screening, or imaging. Anti-idiotypic antibodies and affinity matured antibodies are also considered to be part of the invention.

As used herein, the term “antibody” encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant domains. Each light chain has a variable domain at one end (V(L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (k) and lambda (l), based on the amino acid sequences of their constant domains. Depending on the amino acid sequence of the constant domain of their heavy chains, immunoglobulins can be assigned to different classes. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse or other species. The heavy chain constant domains that correspond to the different classes of immunoglobulins are called alpha, delta, epsilon, gamma, and mu, respectively.

As used herein, the terms “immunoglobulin heavy chain or fragments thereof” and “immunoglobulin light chain or fragments thereof” encompass chimeric peptides and hybrid peptides, with dual or multiple antigen or epitope specificities, and fragments, including hybrid fragments. Thus, fragments of the heavy chains and/or fragments of the light chains that retain the ability to bind their specific antigens are provided. For example, fragments of the heavy chains and/or fragments of the light chains that maintain SIP protein binding activity are included within the meaning of the terms “immunoglobulin heavy chain or fragments thereof” and “immunoglobulin light chain and fragments thereof,” respectively. Such heavy chains and light chains and fragments thereof, respectively, can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

The term “variable” is used herein to describe certain portions of the variable domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies (see Kabat E. A. et al., “Sequences of Proteins of Immunological Interest,” National Institutes of Health, Bethesda, Md. (1987)). The constant domains are not involved directly in binding an antibody to an antigen, but exhibit various effector functions, such as participation of the antibody in antibody-dependent cellular toxicity.

As used herein, the term “antibody or fragments thereof” encompasses chimeric antibodies and hybrid antibodies, with dual or multiple antigen or epitope specificities, and fragments, such as F(ab′)2, Fab′, Fab and the like, including hybrid fragments. Thus, fragments of the antibodies that retain the ability to bind their specific antigens are provided. For example, fragments of antibodies which maintain SIP protein binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies) as described, for example, in U.S. Pat. No. 4,704,692, the contents of which are hereby incorporated by reference.

Optionally, the antibodies are generated in other species and “humanized” for administration in humans. In one embodiment of the invention, the “humanized” antibody is a human version of the antibody produced by a germ line mutant animal. Humanized forms of non-human (e.g., murine) antibodies are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulin. Humanized antibodies include human immunoglobulins (recipient antibody) in which residues from a CDR of the recipient are replaced by residues from a CDR of a non-human species (donor antibody) such as mouse, rat or rabbit having the desired specificity, affinity and capacity. In one embodiment, the present invention provides a humanized version of an antibody, comprising at least one, two, three, four, or up to all CDRs of a SIP monoclonal antibody. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of or at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323-327 (1988); and Presta, Curr. Op. Struct. Biol., 2:593-596 (1992)).

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important in order to reduce antigenicity. According to the “best-fit” method, the sequence of the variable domain of a rodent antibody is screened against the entire library of known human variable domain sequences. The human sequence which is closest to that of the rodent is then accepted as the human framework (FR) for the humanized antibody (Sims et al., J. Immunol., 151:2296 (1993) and Chothia et al., J. Mol. Biol., 196:901 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is important that antibodies be humanized with retention of high affinity for the antigen and other favorable biological properties. To achieve this goal, according to one method, humanized antibodies are prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding (see, WO 94/04679, published 3 Mar. 1994).

Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human antibodies upon antigen challenge (see, e.g., Jakobovits et al., Proc. Natl. Acad. Sci. USA, 90:2551-255 (1993); Jakobovits et al., Nature, 362:255-258 (1993); Bruggemann et al., Year in Immuno., 7:33 (1993)). Human antibodies can also be produced in phage display libraries (Hoogenboom et al., J. Mol. Biol., 227:381 (1991); Marks et al., J. Mol. Biol., 222:581 (1991)). The techniques of Cote et al. and Boerner et al. are also available for the preparation of human monoclonal antibodies (Cole et al., Monoclonal Antibodies and Cancer Therapy, Alan R. Liss, p. 77 (1985); Boerner et al., J. Immunol., 147(1):86-95 (1991)).

Further provided is a hybridoma cell that produces the monoclonal antibody of the invention. An example of such a hybridoma cell is a hybridoma cell which produces a monoclonal antibody that specifically binds an epitope contained within the amino acid sequence identified as SEQ ID NO:2.

The term “monoclonal antibody” as used herein refers to an antibody obtained from a substantially homogeneous population of antibodies, i.e., the individual antibodies comprising the population are identical except for possible naturally occurring mutations that may be present in minor amounts. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired activity (See, U.S. Pat. No. 4,816,567 and Morrison et al., Proc. Natl. Acad. Sci. USA, 81:6851-6855 (1984)).

Monoclonal antibodies of the invention may be prepared using hybridoma methods, such as those described by Kohler and Milstein, Nature, 256:495 (1975) or Harlow and Lane. Antibodies, A Laboratory Manual. Cold Spring Harbor Publications, New York, (1988). In a hybridoma method, a mouse or other appropriate host animal, is typically immunized with an immunizing agent to elicit lymphocytes that produce or are capable of producing antibodies that will specifically bind to the immunizing agent. Alternatively, the lymphocytes may be immunized in vitro. Preferably, the immunizing agent comprises a polypeptide of the present invention. Thus, the disclosed antibodies specifically bind SIP protein, or a fragment thereof. For example, the disclosed antibodies can bind the full-length SIP, or a fragment thereof such as a purified polypeptide fragment of SIP comprising an amino acid sequence identified as SEQ ID NO:2.

Traditionally, the generation of monoclonal antibodies has depended on the availability of purified protein or peptides for use as the immunogen. More recently DNA based immunizations have shown promise as a way to elicit strong immune responses and generate monoclonal antibodies. In this approach, DNA-based immunization can be used, wherein DNA encoding a portion of SIP, for example, expressed as a fusion protein with human IgG1 is injected into the host animal according to methods known in the art (e.g., Kilpatrick K E, et al. Gene gun delivered DNA-based immunizations mediate rapid production of murine monoclonal antibodies to the Flt-3 receptor. Hybridoma. 1998 December; 17(6):569-76; Kilpatrick K E et al. High-affinity monoclonal antibodies to PED/PEA-15 generated using 5 microg of DNA. Hybridoma. 2000 August; 19(4):297-302, which are incorporated herein by reference in full for the methods of antibody production).

An alternate approach to immunizations with either purified protein or DNA is to use antigen expressed in baculovirus. The advantages to this system include ease of generation, high levels of expression, and post-translational modifications that are highly similar to those seen in mammalian systems. Use of this system involves expressing, for example, domains of a SIP antibody as fusion proteins. The antigen is produced by inserting a gene fragment in-frame between the signal sequence and the mature protein domain, for example, of the SIP antibody nucleotide sequence. This results in the display of the foreign proteins on the surface of the virion. This method allows immunization with whole virus, eliminating the need for purification of target antigens.

Generally, either peripheral blood lymphocytes (“PBLs”) are used in methods of producing monoclonal antibodies if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, “Monoclonal Antibodies: Principles and Practice” Academic Press, (1986) pp. 59-103) Immortalized cell lines are usually transformed mammalian cells, including myeloma cells of rodent, bovine, equine, and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells. Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies (Kozbor, J. Immunol., 133:3001 (1984); Brodeur et al., “Monoclonal Antibody Production Techniques and Applications” Marcel Dekker, Inc., New York, (1987) pp. 51-63). The culture medium in which the hybridoma cells are cultured can then be assayed for the presence of monoclonal antibodies directed against SIP. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art, and are described further in the Examples below or in Harlow and Lane “Antibodies, A Laboratory Manual” Cold Spring Harbor Publications, New York, (1988).

After the desired hybridoma cells are identified, the clones may be subcloned by limiting dilution or FACS sorting procedures and grown by standard methods. Suitable culture media for this purpose include, for example, Dulbecco's Modified Eagle's Medium and RPMI-1640 medium. Alternatively, the hybridoma cells may be grown in vivo as ascites in a mammal. The monoclonal antibodies secreted by the subclones may be isolated or purified from the culture medium or ascites fluid by conventional immunoglobulin purification procedures such as, for example, protein A-Sepharose, protein G, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, plasmacytoma cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567) or by covalently joining to the immunoglobulin coding sequence all or part of the coding sequence for a non-immunoglobulin polypeptide

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994, U.S. Pat. No. 4,342,566, and Harlow and Lane, Antibodies, A Laboratory Manual, Cold Spring Harbor Publications, New York, (1988). Papain digestion of antibodies typically produces two identical antigen binding fragments, called Fab fragments, each with a single antigen binding site, and a residual Fc fragment. Pepsin treatment yields a fragment, called the F(ab′)2 fragment, that has two antigen combining sites and is still capable of cross-linking antigen.

The Fab fragments produced in the antibody digestion also contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)2 fragment is a bivalent fragment comprising two Fab′ fragments linked by a disulfide bridge at the hinge region. Fab′-SH is the designation herein for Fab′ in which the cysteine residue(s) of the constant domains bear a free thiol group. Antibody fragments originally were produced as pairs of Fab′ fragments which have hinge cysteines between them. Other chemical couplings of antibody fragments are also known.

An isolated immunogenically specific paratope or fragment of the antibody is also provided. A specific immunogenic epitope of the antibody can be isolated from the whole antibody by chemical or mechanical disruption of the molecule. The purified fragments thus obtained are tested to determine their immunogenicity and specificity by the methods taught herein. Immunoreactive paratopes of the antibody, optionally, are synthesized directly. An immunoreactive fragment is defined as an amino acid sequence of at least about two to five consecutive amino acids derived from the antibody amino acid sequence.

Further provided are fragments of antibodies which have bioactivity. The polypeptide fragments can be recombinant proteins obtained by cloning nucleic acids encoding the polypeptide in an expression system capable of producing the polypeptide fragments thereof, such as an adenovirus or baculovirus expression system. For example, one can determine the active domain of an antibody from a specific hybridoma that can cause a biological effect associated with the interaction of the antibody with a SIP polypeptide. For example, amino acids found not to contribute to either the activity or the binding specificity or affinity of the antibody can be deleted without a loss in the respective activity. For example, in various embodiments, amino or carboxy-terminal amino acids are sequentially removed from either the native or the modified non-immunoglobulin molecule or the immunoglobulin molecule and the respective activity assayed in one of many available assays. In another example, a fragment of an antibody comprises a modified antibody wherein at least one amino acid has been substituted for the naturally occurring amino acid at a specific position, and a portion of either amino terminal or carboxy terminal amino acids, or even an internal region of the antibody, has been replaced with a polypeptide fragment or other moiety, such as biotin, which can facilitate in the purification of the modified antibody. For example, a modified antibody can be fused to a maltose binding protein, through either peptide chemistry or cloning the respective nucleic acids encoding the two polypeptide fragments into an expression vector such that the expression of the coding region results in a hybrid polypeptide. The hybrid polypeptide can be affinity purified by passing it over an amylose affinity column, and the modified antibody receptor can then be separated from the maltose binding region by cleaving the hybrid polypeptide with the specific protease factor Xa. (See, for example, New England Biolabs Product Catalog, 1996, pg. 164.). Similar purification procedures are available for isolating hybrid proteins from eukaryotic cells as well.

The fragments, whether attached to other sequences or not, include insertions, deletions, substitutions, or other selected modifications of particular regions or specific amino acids residues, provided the activity of the fragment is not significantly altered or impaired compared to the nonmodified antibody or antibody fragment. These modifications can provide for some additional property, such as to remove or add amino acids capable of disulfide bonding, to increase its bio-longevity, to alter its secretory characteristics, etc. In any case, the fragment must possess a bioactive property, such as binding activity, regulation of binding at the binding domain, etc. Functional or active regions of the antibody may be identified by mutagenesis of a specific region of the protein, followed by expression and testing of the expressed polypeptide. Such methods are readily apparent to a skilled practitioner in the art and can include site-specific mutagenesis of the nucleic acid encoding the antigen. (Zoller M J et al. Nucl. Acids Res. 10:6487-500 (1982).

A variety of immunoassay formats may be used to select antibodies that selectively bind with a particular protein, variant, or fragment. For example, solid-phase ELISA immunoassays are routinely used to select antibodies selectively immunoreactive with a protein, protein variant, or fragment thereof. See Harlow and Lane. Antibodies, A Laboratory Manual. Cold Spring Harbor Publications, New York, (1988), for a description of immunoassay formats and conditions that could be used to determine selective binding. The binding affinity of a monoclonal antibody can, for example, be determined by the Scatchard analysis of Munson et al., Anal. Biochem., 107:220 (1980).

Also provided is an antibody reagent kit comprising containers of a monoclonal antibody or fragment thereof of the invention and one or more reagents for detecting binding of the antibody or fragment thereof to a SIP polypeptide. The reagents can include, for example, fluorescent tags, enzymatic tags, or other tags. The reagents can also include secondary or tertiary antibodies or reagents for enzymatic reactions, wherein the enzymatic reactions produce a product that can be visualized. Thus, provided is a kit comprising the disclosed antibodies.

Provided is a method of increasing production of a steroid hormone in a subject in need thereof, comprising administering to the subject an effective amount of a purified polypeptide fragment of a steroidogenesis inducing protein (SIP) having fewer amino acids than the entire steroidogenesis inducing protein, comprising an amino acid sequence identified as SEQ ID NO:2, and a pharmaceutically acceptable carrier. As used herein, an “effective amount” refers to an amount that is administered to a subject that is sufficient to achieve the desired therapeutic result or to have an effect on undesired symptoms, but is generally insufficient to cause adverse side affects. In one aspect, the purified polypeptide fragment has an amino acid sequence greater than about 95% similar to an amino acid sequence identified as SEQ ID NO:2. In another aspect, the purified polypeptide fragment consists essentially of an amino acid sequence identified as SEQ ID NO:2. In yet another aspect, the purified polypeptide fragment consists of an amino acid sequence identified as SEQ ID NO:2.

The dosage ranges for the administration of the compositions are those large enough to produce the desired effect in which the symptoms are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any contraindications. Dosage can vary, and can be administered in one or more dose administrations daily, for one or several days. An effective amount of the disclosed purified polypeptides can be from about 0.1 μg to about 5 μg administered to the subject four to six times per day. In another aspect, the effective amount can be from about 1 μg to about 3 μg per day. A subject can receive a single injection, or, if additional injections are necessary, they can be repeated at other appropriate time intervals, as determined by the skilled practitioner, for an indefinite period and/or until the efficacy of the treatment has been established.

The compositions may be administered orally, parenterally (e.g., intravenously, subcutaneously, by intramuscular injection), by intraperitoneal injection, intracavity, intrathecally, transdermally, extracorporeally, rectally, directly into a targeted tissue or organ (e.g., testes), topically, or the like. As used herein, “topical intranasal administration” means delivery of the compositions into the nose and nasal passages through one or both of the nares and can comprise delivery by a spraying mechanism or droplet mechanism, or through aerosolization of the composition. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the disorder being treated, the particular composition and its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycled to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

In one aspect, the disclosed compositions and a pharmaceutically acceptable carrier can be administered to a subject transdermally. For example, a transdermal patch containing a composition can be applied to the skin of the scrotum in a male subject who is in need of producing more testosterone in his testes. Alternatively, a person of skill can administer the composition directly into a subject's testes to induce testosterone production. In another aspect, the composition can be administered intraperitoneally to induce increased production of progesterone, testosterone, and estrogen in the ovaries of a female subject in need thereof. In another aspect, the composition can be administered intraperitoneally to induce increased production of glucocorticoids, mineralocorticoids and androgens in the adrenal glands of a subject in need thereof.

The disclosed polypeptides in a pharmaceutically acceptable carrier can be coupled to either human serum albumen or the constant region of an immunoglobulin heavy chain which does not elicit an immune reaction and administered to a subject in need thereof. For delivery to the testes or ovaries in a subject, the disclosed compositions can be fused to antibodies to LH and FSH receptors. For delivery to adrenal glands in a subject, the disclosed compositions can be fused to antibodies to ACTH. For example, a fusion protein comprising an immunoglobulin heavy chain and a polypeptide fragment comprising an amino acid sequence identified as SEQ ID NO:2 can be used. In one aspect, a fusion protein comprising an immunoglobulin heavy chain and a polypeptide fragment comprising an amino acid sequence greater than about 95% similar to an amino acid sequence identified as SEQ ID NO:2 can be used. In another aspect, a fusion protein comprising an immunoglobulin heavy chain and a polypeptide fragment consisting essentially of an amino acid sequence identified as SEQ ID NO:2 can be used. In another aspect, a fusion protein comprising an immunoglobulin heavy chain and a polypeptide fragment consisting of an amino acid sequence identified as SEQ ID NO:2 can be used. For delivery to prostate gland cancer cells or ovarian cancer cells in a subject, the disclosed compositions can be fused to a protein which specifically binds to a membrane protein in the cancer cell.

Further provided is a method of increasing conversion of cholesterol to pregnenolone in a cell, comprising contacting the cell with a purified polypeptide fragment of a steroidogenesis inducing protein having fewer amino acids than the entire steroidogenesis inducing protein, comprising an amino acid sequence identified as SEQ ID NO:2, whereby contacting the cell increases conversion of cholesterol to pregnenolone in the cell. By increasing the conversion of cholesterol into pregnenolone in a cell, the disclosed polypeptides affect the rate-limiting step in the pathway of steroid production in a target cell. For example, by increasing the level of pregnenolone in a testicular cell, for example in a Leydig cell, the disclosed purified polypeptides can increase the production and secretion of testosterone in the testes of a male subject. By increasing the level of pregnenolone in an ovarian cell, for example a granulosa cell, a theca cell, or a luteal cell, the disclosed purified polypeptides can increase the production and secretion of progesterone and estrogen in the ovaries of a female subject. In another aspect, by increasing the level of pregnenolone in an adrenal gland cortex cell, the disclosed purified polypeptides can increase the production and secretion of glucocorticoids, mineralocorticoids and androgens in the adrenal glands of a subject in need thereof.

Also provided is a method of inhibiting the elaboration of steroidogenesis inducing protein (SIP) from a cell in a subject, comprising blocking the interaction of the cell with extracellular SIP, comprising contacting the extracellular SIP with an agent that binds to the extracellular SIP at a site of contact between the extracellular SIP and a cell membrane-bound receptor, thereby inhibiting interaction between the extracellular SIP with the receptor and inhibiting elaboration of SIP from the cell of the subject. SIP is naturally produced and is secreted by various tissues, for example, the ovary, testis, and adrenal gland. As used herein, “extracellular SIP” is SIP that is naturally produced in a cell of a subject and secreted from the cell into the extracellular space surrounding the cell.

Extracellular SIP also acts in an autocrine manner to stimulate proliferation of nearby cells by interacting with membrane-bound receptors on cells which leads to cell division and proliferation of the cells. After binding to the membrane receptors, SIP activates tyrosine kinase activity associated with the SIP receptors and other intracellular proteins. This cascade leads to the activation of RAS proteins which cause the activation of MAP-kinase pathway. The SIP signaling also results in the activation of PI3-kinase leading to the activation of Protein kinase B/Akt pathway. Both of these pathways are required for SIP effects on cell proliferation. Most of the growth factors which stimulate proliferation in a wide variety of cells use one or both of these pathways for their effects. Examples of an agent that can bind to extracellular SIP at a site of contact between the extracellular SIP and a cell membrane-bound receptor include, but are not limited to, an antibody, a peptide antagonist, or a synthetic compound. In one aspect, the agent can be an antibody that specifically binds the amino acid sequence identified as SEQ ID NO:2 which is contained in SIP. The antibody can be polyclonal or monoclonal.

Also provided is a method of inhibiting the proliferation of a cancer cell, comprising blocking the interaction of the cell with extracellular SIP, comprising contacting the extracellular SIP with an agent that binds to the extracellular SIP at a site of contact between the extracellular SIP and a cell membrane-bound receptor, thereby inhibiting interaction between the extracellular SIP with the receptor and inhibiting proliferation of the cell. Inhibiting the proliferation of a cancer cell can occur in vitro and in vivo. Examples of a cancer include, but are not limited to, ovarian cancer, testicular cancer, prostate cancer, breast cancer, and liver cancer.

Further provided is a method of screening for a compound that inhibits the interaction of extracellular SIP and a cell membrane-bound receptor of SIP, comprising the steps of: a) contacting the extracellular SIP with the compound; b) contacting a cell that expresses the cell membrane-bound receptor of SIP with the extracellular SIP; and c) determining whether the extracellular SIP of step a) is inhibited from binding to the membrane-bound receptor of SIP. Inhibition of binding between the extracellular SIP and the membrane-bound receptor is indicated by a reduction in SIP elaboration from the cell.

Also provided is a method of detecting SIP in a body fluid of a subject, comprising contacting the fluid with an antibody that specifically binds to a polypeptide fragment comprising an amino acid sequence identified as SEQ ID NO:2 under conditions wherein the antibody can bind to SIP, wherein the detection of SIP/antibody complexes indicates the presence of SIP in the body fluid of the subject. Examples of a body fluid include, but are not limited to, blood, plasma, serum, mucus, urine, semen, lymph, milk, saliva, cerebrospinal fluid, perspiration, and ascites fluid. Detecting SIP in biological fluids can be used as a method for screening cancer in a subject. SIP is produced in high amounts in cancer patients and may accumulate in body fluids. In subjects without cancer, secretion of SIP will be low and restricted to the tissues where it performs an autocrine function (e.g., in testes, ovaries, and adrenal glands).

### EXPERIMENTAL

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Chemicals and Reagents

Recombinant human EGF was purchased from R&D systems (Minneapolis, Minn.). Ovine LH was obtained from NIDDK (Bethesda, Md.). The antibodies against Erk1/2, Mek1/2, pan-Erk and Akt were purchased from Cell Signaling Technology (Beverly, Mass.). Rabbit anti-steroidogenic acute regulatory (StAR) protein antiserum was a gift from Dr. D. Stocco (Lubbock, Tex.).

### Purification of SIP by Chromatography

Human follicular fluid (hFF) was obtained at the time of egg retrieval from women participating in the in vitro fertilization (IVF) programs. These women had been treated with human menopausal gonadotropin (hMG) and human chorionic gonadotropin (hCG) to induce follicular development and ovulation, respectively. Pooled, cell-free hFF was heated at 60° C. for 15 mM to inactivate possible residual proteolytic enzymes and then centrifuged at 1500×g for 10 mM This heating step had negligible effects on SIP bioactivity. hFF proteins were then precipitated with 80% ammonium sulfate at 4° C. overnight. Precipitates were dissolved in 15 mM Tris-HCl (pH 7.2) and dialyzed for 48 hrs, followed by precipitation with 10 volumes of acetone to remove remaining steroids. Precipitates were re-dissolved in Tris-HCl buffer. The hFF proteins were then subjected to gel chromatography on a Sephacryl S-200 column (Pharmacia; dimensions 2.5×80 cm) as described previously (10). Pooled bioactive fractions were concentrated using Centriprep centrifugal concentrators (Amicon; MW cut of 10,000) and further purified by Cibacron-Blue affinity chromatography (Blue Sepharose, Pharmacia; column dimensions 1.0×30 cm). Proteins were loaded on the column dissolved in Tris-HCl buffer (pH 7.2), and the unbound proteins were eluted in the initial buffer. The proteins which bound to the Blue Sepharose column were eluted in two steps: 20 mM Tris-HCl buffer (pH 7.2), containing 0.15 M NaCl, was used to elute the first fraction of the bound proteins, while the rest of the bound proteins were eluted using the above buffer containing 2M NaCl. SIP bioactivity was present in both bound fractions. However, the fraction eluted with 0.15 M NaCl was used for further purification because of relatively higher amounts of human serum albumin (hSA) in fractions eluted with 2 M NaCl Tris-HCl buffer. After concentration by Amicon filtration as described above, the pooled bioactive fractions were then applied to a Heparin Sepharose column (Pharmacia; dimensions 1×1.5 cm) to remove possible heparin-binding proteins such as those of the EGF and FGF family, and β₂-macroglobulin. SIP bioactivity was eluted in the unbound protein fractions, which were pooled and concentrated. These fractions still contained a significant amount of hSA, which was removed by an additional chromatography on Blue Sepharose. SIP preparations were applied on a small Blue Sepharose column (1×5 cm) in Tris-HCl buffer containing 0.15 M NaCl. SIP was eluted in the unbound fractions while hSA and other remaining bound proteins were eluted with Tris-HCl buffer containing 2 M NaCl. In some experiments chromatofocusing was used to purify SIP using a PBE-94 column (1×1.5 cm; Pharmacia). This technique proved to be very useful for complete removal of hSA from partially purified SIP preparation (12). Proteins were loaded onto the PBE column in 0.25 M triethyleneamine buffer (pH 9.4). SIP bioactivity was eluted in the unbound fraction. The bulk of bound proteins including hSA were then eluted with Tris-HCl buffer (pH 6.0). The unbound protein fractions were concentrated and equilibrated into 20 mM Tris-HCl buffer (pH 7.2) by Sephadex G25 chromatography (NAP 10; Pharmacia).

### Protein Sequencing

The purified SIP preparations were mixed with 2× Lammeli's sample buffer (125 mM Tris-HCl, pH 6.8, 20% glycerol, 4% SDS, 10% β-mercaptoethanol, and 0.05% Bromophenol blue) and subjected to SDS-PAGE in 8% gels, and the protein bands were identified by Coomassie blue or silver staining. To assess which of the protein band(s) exhibited SIP bioactivity, the purified SIP fractions were run on 8% SDS-PAGE gels, the protein bands were visualized by 0.25 M KCl, and the visible bands were excised from the gel. Bioactivity was determined after removal of SDS and renaturation of proteins (6). The protein band corresponding to the one exhibiting SIP bioactivity was used for N-terminal amino acid sequence analysis by an automated amino acid sequencer (Protein sequencer, 477 A; Applied Biosystems, Wurlington, Calif.) after separation on SDS-PAGE and transfer to PVDF membrane (Millipore). Also, the purified SIP fractions were analyzed by Mass Spectrometry and additional peptide sequences were obtained.

### Synthesis of SIP Peptide

A purified polypeptide containing 17 amino acids (SEQ ID NO:2) was synthesized, and polyclonal antisera were raised against this polypeptide using commercial sources. A jumbled polypeptide (SEQ ID NO:1) was synthesized using same commercial source and maintained and tested in our biological assays under identical conditions.

### Isolation and Culture of Immature Leydig Cells

Immature rat Leydig cells (ILC) were isolated from 10-day old rat testes and cultured with serum-free medium before treatment. Immature rats were sacrificed at 10 days of age, and the testes were removed Immature Leydig cells were isolated and cultured. The decapsulated testes were incubated for 5 min in culture medium containing 0.25 mg collagenase/ml (Type I, Sigma) in shaking water bath. The seminiferous tubules were allowed to settle at unit gravity for 5 min, and the supernatant containing the suspension of interstitial cells was removed. The supernatant was centrifuged at 90×g for 10 min, and the cell pellet was washed twice by re-suspension in fresh medium followed by centrifugation. The cell suspension was plated in 24-well tissue culture plates (Nunclon; Nunc, Roskilde, Denmark; 2×10⁵ cells/well) or in 6-well plates (2×10⁶ cells/well) depending upon the end point of the experiment. The cells were cultured at 37° C. in a humidified atmosphere of 5% CO₂ and 95% air in serum-free medium. After 1 h, the immature Leydig cells were well attached to the surface of the culture plates, whereas contaminating cells (Sertoli cells, germ cells, precursor Leydig cells) were loosely attached. The cells were washed three times with medium to remove loosely attached cells. At this stage, the cell preparation contains more than 90% immature Leydig cells. Immature Leydig cells cultured in 24-well plates (2×10⁵ cells/well) were treated with several doses of oLH for 48 h, and the conditioned media were analyzed for androgen content.

### Culture of Ovarian, Prostate and Breast Cancer Cells

Ovarian (HEY, SKA), prostate (DU145, PC3) and breast (MCF7) cancer cell lines were cultured and maintained using well-established culture procedures.

### Thymidine Incorporation Assay

Immature rat Leydig cells and various cancer cell lines were starved for 48 hours before treatment. The cells were then treated with SIP, the polypeptide having the amino acid sequence identified as SEQ ID NO:2, LH, and EGF for 18 hours. The incubation media were collected (Leydig cells) and analyzed for total androgen production. ³H-thymidine (1 μCi/ml) was added to the cells for further 4 hours incubation. Then the hot media were removed; ice-cold water was added to the cells which were lysed by sonication. The cell lysates were filterd through DE81 ion-exchanger filters. The filter membranes were then put in vials filled with scintillation fluid, and the radioactivity was measured by a Beckman β-counter.

### Steroidogenesis

Immature Leydig cells were cultured for 18 h and treated with different concentrations of oLH, SIP and EGF. For some experiments, the cells were treated with oLH or SIP for 18 h in the absence or presence of inhibitors of tyrosine kinase (Genistein; 10 uM), MAPK-kinase (PD98059; 25 μM), PI3-kinase (LY294002; 5 μM) or PKA (H89; 10 μM) to determine the intracellular signaling pathways involved in the effects of SIP on androgen production. The incubation media were collected and analyzed for total androgens using a radioimmunoassay, as described previously (Dickson et al., 2002). Immature rat Leydig cells predominantly secrete 5α-reduced androgens (Benton et al., 1995). Therefore, to determine the total androgens produced during the incubation period, an antiserum which exhibits 77% and 26% crossreactivity to 5α-dihydrotestosterone and 5α-androstane, 3α, 17β diol, respectively was employed (Ismail et al., 1972). All samples were analyzed in the same assay, and the intra-assay coefficient of variation was <10%.

### Western Blot Analyses

To determine the effects of SIP and EGF on activation of proteins involved in either MAP-kinase or PI3-kinase signaling pathway, immature Leydig cells were cultured in 6-well plates. The cells were washed and treated with SIP (50 μg/ml) or EGF (10 ng/ml) for 30 min in the presence or absence of inhibitors of MAPK-kinase (PD98059; 25 μM) or PI3-kinase (LY294002; 5 μM). The cells were washed twice with ice-cold phosphate-buffered saline and lysed in lysis buffer (Cell Signaling Technology, Beverly, Mass.) containing 20 mM Tris-HCl (pH 7.5), 150 mM NaCl, 1 mM Na2EDTA, 1 mM EGTA, 1% Triton, 2.5 mM sodium pyrophosphate, 1 mM β-glycerophosphate, 1 mM Na3VO4, 1 μg/ml leupeptin and 1× protease inhibitor cocktail (Calbiochem, San Diego, Calif.). Protein concentrations were determined by the Lowry HS assay using the Bio-Rad DCProtein Assay kit (Bio-Rad, Hercules, Calif.) according to the instruction provided by the manufacturer. Cell lysates were mixed with Lammeli's buffer (62.5 mM Tris, pH 6.8, 2% SDS, 5% β-mercaptoethanol and 10% glycerol), and individual samples (25 μg protein) were subjected to SDS-PAGE in 10% gels and transferred to PVDF membranes (Millipore). The membranes were blocked overnight at 4° C. in TBST (50 mM Tris, pH 7.5, containing 0.15M NaCl, 0.05% Tween-20 and 4% fat-free skimmed milk). The blots were then incubated with appropriate dilutions of specific primary antibodies for 1 h at room temperature. After washing, the blots were incubated with anti-rabbit or anti-mouse immunoglobulin coupled to horseradish peroxidase (dilution 1:5000; Amersham) for 1 h. The blots were then developed in ECL mixture (Pierce) for 1 mM, exposed to an X-ray film and visualized by autoradiography. Western blots for β-actin were carried out in parallel as internal controls.

RNA Isolation, cDNA Synthesis and RT-PCR

Total RNA was isolated from Leydig cells using TRIzol (Invitrogen, Carlsbad, Calif.) followed by chloroform extraction and isopropanol precipitation. Two micrograms of total RNA were reverse transcribed at 37° C. for 1 h in a reaction mixture containing 0.5 mM dNTP (Fisher Scientific), 400 U of M-MLV Reverse Transcriptase (Promega), 1×RT buffer, 0.5 mM dithiotreitol (Bio-Rad) and 0.5 μg of oligo dT (Promega). After incubation, the RT enzyme was heat inactivated at 70° C. for 5 mM and the reaction cooled to 4° C. Four microliters of RT reactions were added to separate PCR reaction mixtures (total 10 μl) containing 0.1 mM dNTPs, (Fisher), 0.5 U Tag DNA polymerase (PGC Scientifics, Frederick, Md.), 1×PCR buffer with 3 mM MgC12 (Idaho Technology, Salt Lake City, Utah) and 25 μmol of the specific primers for the following genes: scavenger receptor B type 1 (SR-B1), cytochrome P450 cholesterol side-chain cleavage (P450scc), 3β-hydroxysteroid dehydrogenase (3β-HSD), 17 alpha-hydroxylase/C17-20 lyase (P450c17) and steroidogenic acute regulatory (StAR) protein. The primer pairs and expected products for individual genes are shown in Table 2. L19 (a ribosomal protein) primer pair was used as internal control. The following protocol was used in the Rapid Cycler thermal cycler (Idaho Technology): 94° C. for 15 s (initial denaturation), followed by 94° C. for 0 s, 60° C. for 0 s, 72° C. for 20 s for 35 cycles, and 72° C. for 30 s (final extension). The oligonucleotide pairs for RT-PCR of SR-B1, StAR, P450scc, 3β-HSD, P450c17 and L-19 were synthesized according to previously published reports (Shultz et al., 2001; Tena-Sempere et al., 2002; Watch and Morris, 2002). The PCR products were visualized on 1%-2% agarose gels stained with ethidium bromide. Relative amounts of PCR products were estimated by digital densitometry with FlourChem (Alpha Innotech Corporation, San Leandro, Calif.). The expression of selected genes was normalized relative to the expression of L19.

### Results

### Purification and Partial Amino Acid Analysis of SIP

SIP from human ovaries was purified. The partial amino acid sequence of the purified protein was determined. The sequences for an N-terminal peptide 1 (8 amino acids (SEQ ID NO:1)); and two endogenous peptides, i.e., peptide 2 (17 amino acids (SEQ ID NO:2)); and peptide 3 (14 amino acids (SEQ ID NO:3)), have been determined and reported in protein databases. The sequences for peptide 1 and peptide 3 were found in a group of cDNA which have been isolated from several human tissues and showed similarities to immunoglobulins. However, the sequence of peptide 2 (SEQ ID NO:2) was not present in any of these proteins. The peptide 2 sequence showed significant homologies with another group of proteins (DING proteins) which have been purified from several different sources; however, none of these proteins has been cloned, and the sequences have not been found in any genomic data bases.

### Generation of Anti-SIP Antibodies

Using the sequences determined after purification of SIP, anti-SIP antibodies were raised. Peptide 2 (SEQ ID NO:2) was selected for this purpose because this peptide was not found in proteins of immunoglobulin family. Hence, this peptide is specific to SIP. A 17 amino acid peptide was synthesized, and antibodies against this peptide were raised in rabbit using commercial sources.

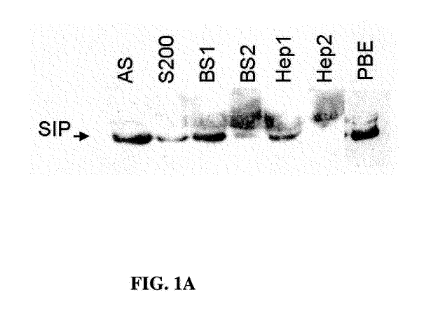

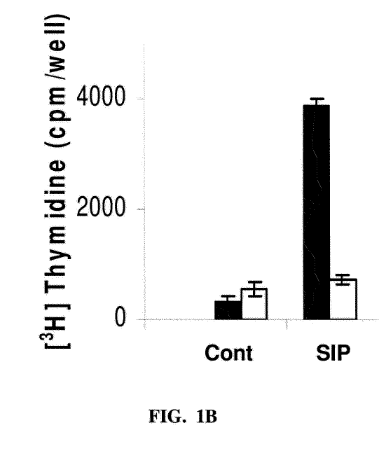

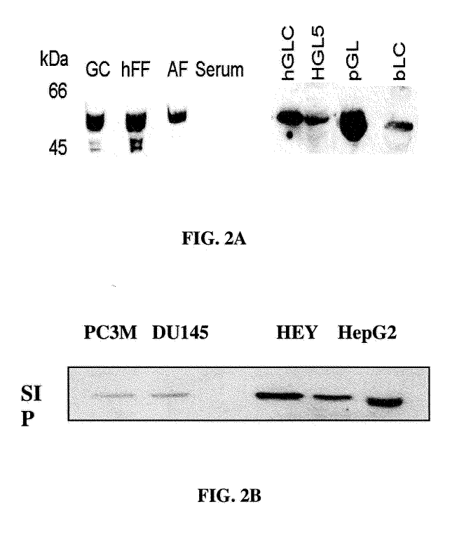

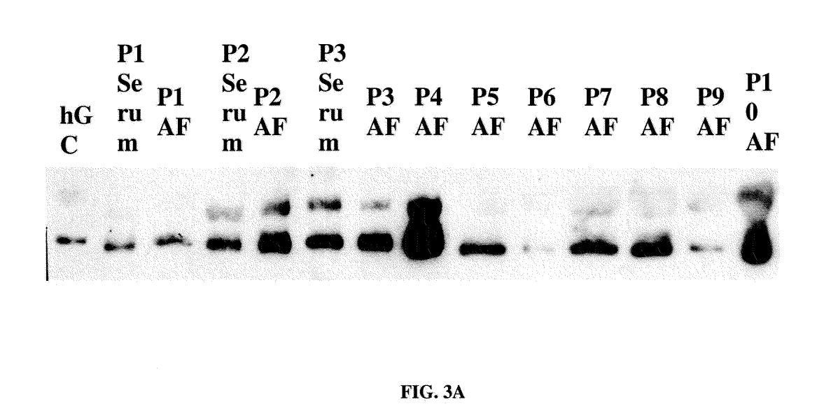

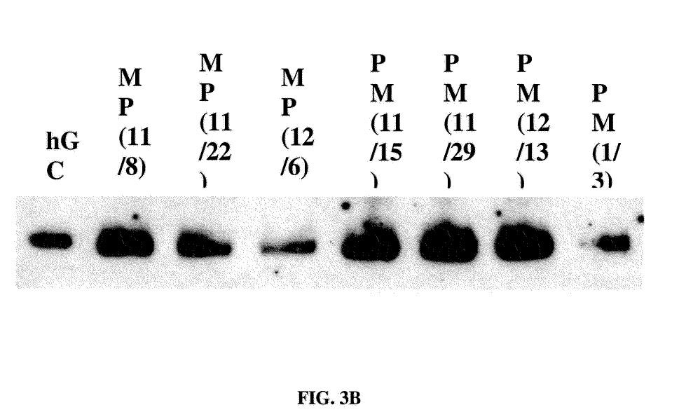

The antiserum against the polypeptide having the amino acid sequence identified as SEQ ID NO:2 was characterized for its specificity for SIP detection. These studies revealed that the antibodies are specific for a 50 kD protein which is found in all purified SIP fractions. These antibodies also neutralized the biological activity of SIP (FIG. 1).

Further analysis also revealed that similar proteins are secreted by human ovarian cells, ovarian, prostate and liver cancer cells, as determined by Western blotting. These results suggested that several normal and transformed cells secrete SIP, which serves as an autocrine growth factor for these cells. Further, purified SIP stimulates proliferation of these cells in vitro. These studies indicated that peptide 2 (SEQ ID NO:2) is part of the biologically active SIP protein and that this protein is secreted by normal ovaries and also by ovarian and prostate cancer cells.

### Effects of Synthetic Peptide on DNA Synthesis

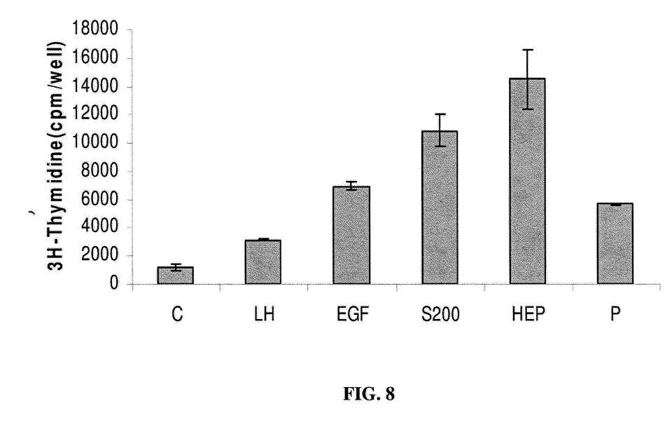

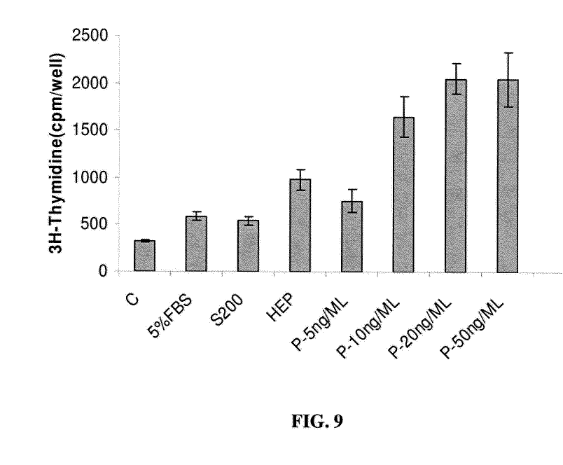

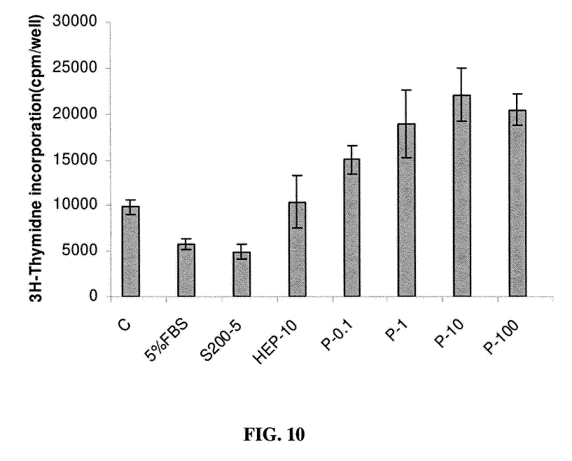

Because peptide 2 (SEQ ID NO:2) is specific to the SIP protein and is not found in other members of SIP-like protein family, it was thought that this peptide sequence is essential for SIP biological activity. Therefore, this novel polypeptide was tested for its effects on DNA synthesis in immature rat Leydig cells and on ovarian, prostate and liver cancer cell lines. These results are presented in FIGS. 8-10. The data showed that like the purified SIP protein, the polypeptide fragment (identified as having an amino acid sequence of SEQ ID NO:2) caused significant stimulation of DNA synthesis in different cells. These data suggested that this synthetic polypeptide contains the sequences required for binding to SIP receptors and induction of its proliferatory effects.

### Effects of Synthetic Peptide 2 (SEQ ID NO:2) on Steroidogenesis

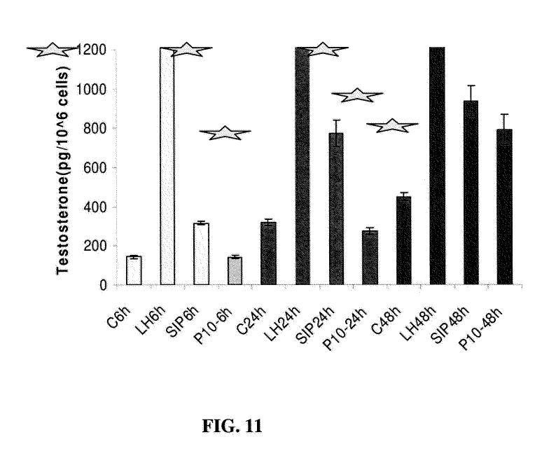

Synthetic peptide 2 was tested to determine whether, like SIP, it stimulates steroid production in target cells. Therefore, the effects of SIP and the polypeptide fragment of SIP (SEQ ID NO:2) on testosterone production by immature rat Leydig cells were tested. As shown in FIG. 11, both SIP and SEQ ID NO:2 caused significant stimulation of testosterone production in Leydig cell cultures.

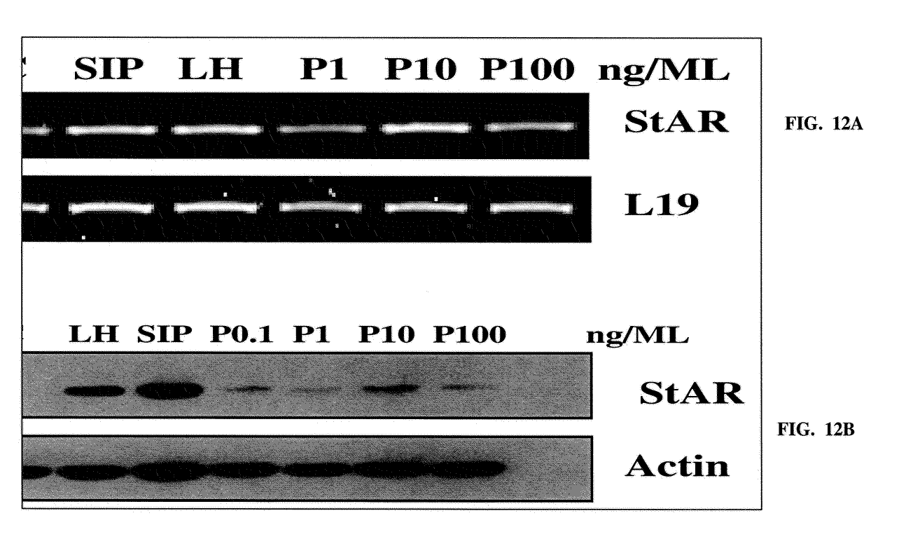

These studies were extended to identify the steps at which peptide 2 (SEQ ID NO:2) may effect the increase in testosterone production. As shown in FIG. 12, SIP protein and synthetic peptide 2 both stimulated the induction of StAR protein in Leydig cells. StAR protein is required for the transport of cholesterol into inner membrane of mitochondria which is a rate-limiting step in steroid production in all steroidogenic tissues.

### Mechanism of Action of SIP and Synthetic Peptide 2 (SEQ ID NO:2)

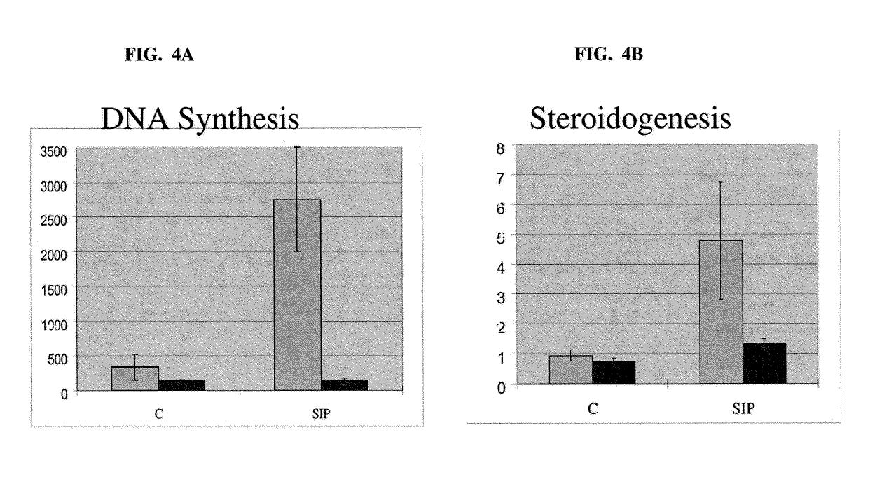

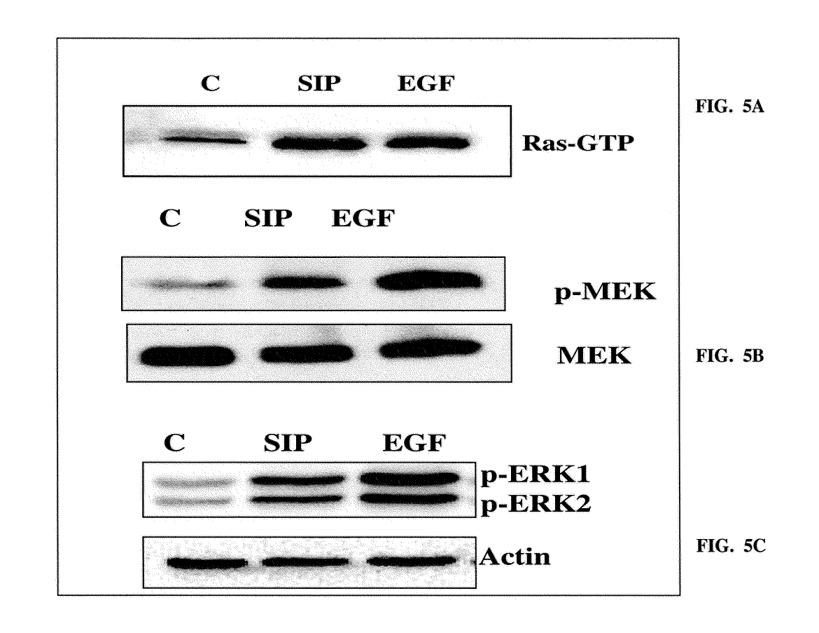

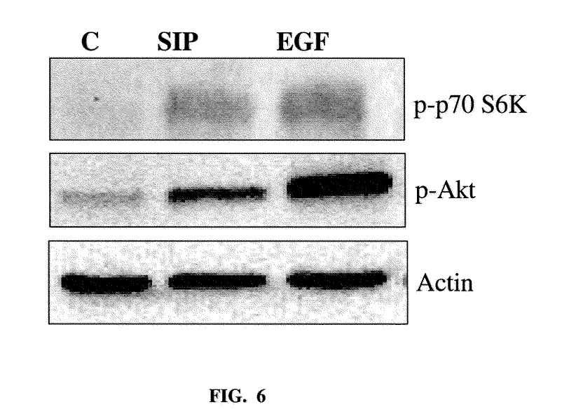

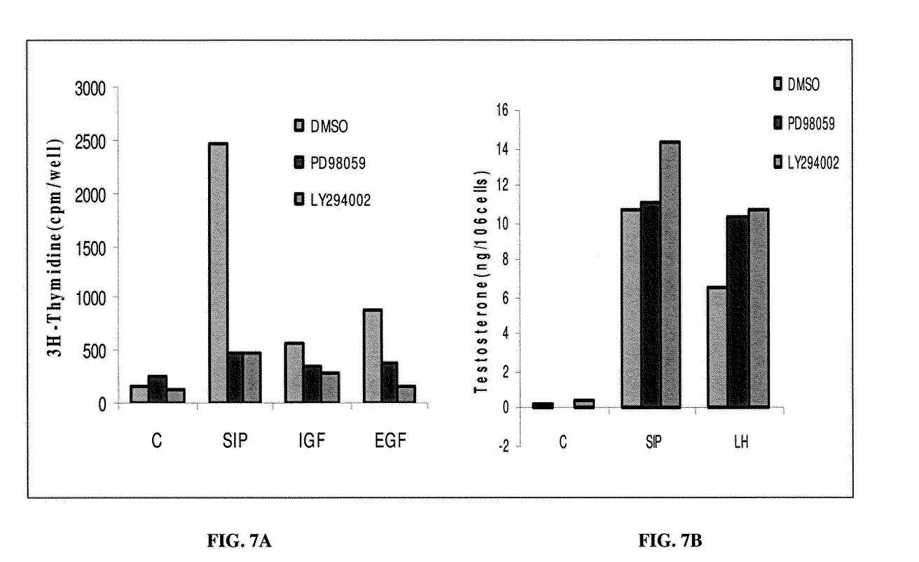

Previous studies have indicated that SIP effects on target cells are mediated by a tyrosine kinase coupled signaling pathway (FIG. 4). Stimulation of immature Leydig cells and ovarian cancer cells results in a rapid increase in tyrosine phosphoryation of several proteins which may be involved in SIP effects on target cells. Further studies indicated that, like several other growth factors, SIP stimulates the activation of MAP/ERK kinase and PI3-kinase/AKT pathways in immature Leydig cells (FIGS. 5-7). Both of these pathways are required for the mitogenic effects of SIP on these cells; however, these pathways are not involved in its effects on steroid production.

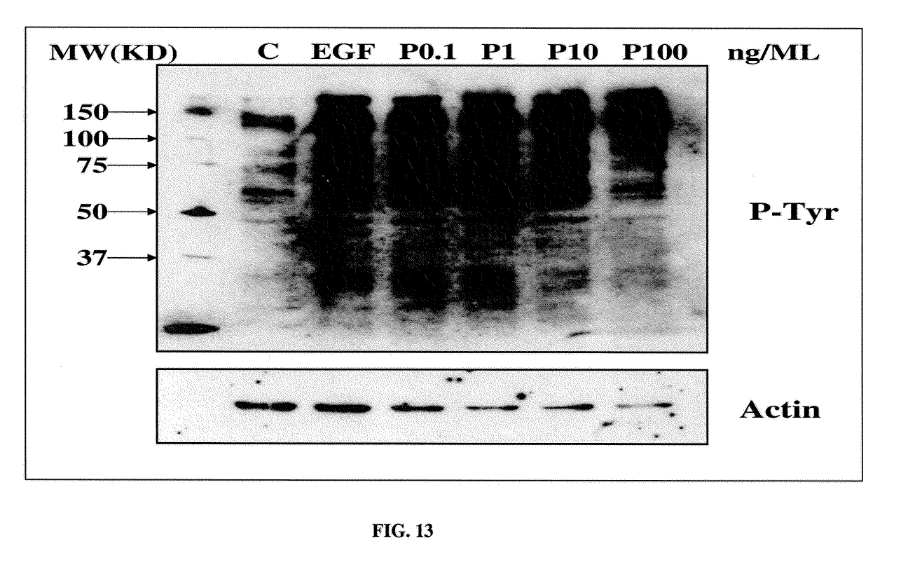

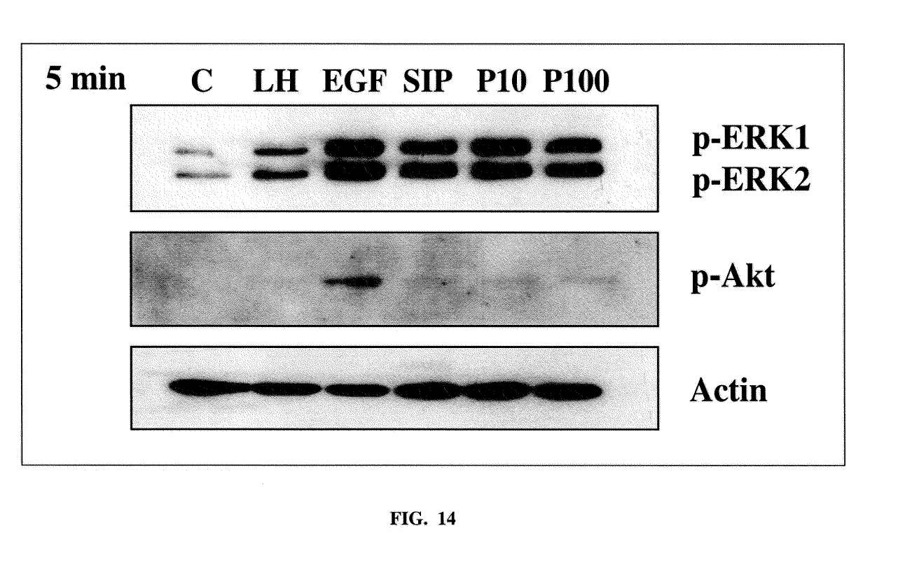

Because synthetic peptide 2 (SEQ ID NO:2) exerts positive effects on proliferation and steroidogenesis which are qualitatively similar to those observed with purified SIP, the effects on cell signaling components in immature rat Leydig cells were investigated. As shown in FIG. 13, synthetic peptide 2 induces the phosphorylation of several proteins at tyrosine in immature Leydig cells similar to those observed with the purified SIP. Furthermore, the synthetic peptide also caused a significant induction of phosphorylation of ERK1 and ERK2 and AKT in these cells similar to the effects of purified SIP (FIG. 14). These studies indicate that the synthetic peptide is capable of initiating signaling pathways and exert biological effects on proliferation and steroidogenesis in target cells which are similar to those exerted by the purified 50 kD SIP protein. Hence, this 17 amino acid polypeptide can bind to SIP receptor and induce SIP effects on the target cells.

Ovarian and prostate cancer cells produce SIP. When SIP is added to cultures of ovarian or prostate cancer cells, the cells undergo increased cell proliferation. These results indicate that ovarian and prostate tumors produce this protein which is required for their continuous growth. Therefore, blockage of SIP production and/or inhibition of its effects will result in the reduction of tumor size in both ovarian and prostate cancer patients. Antibodies that specifically bind synthetic peptide 2 (SEQ ID NO:2) were raised. These antibodies neutralize the effects of SIP on cell proliferation and steroid production in testicular cells. Further, antagonists of the 17 amino acid peptide (SEQ ID NO:2) block the interaction of SIP with its receptors.

### In Vitro Experiments

Ovarian (HEY, SKA) and prostate cancer (DU145 and PC3) cells are cultured under optimal growth conditions in 24 well plates. The cells are serum starved for 48 h and are treated with SIP or synthetic peptide 2 (SEQ ID NO:2) in the presence or absence of antibodies or peptide antagonists for 18 hours. The doses for SIP and peptide 2 are the same as used in the previous studies. The antiserum is used at 1:1000 dilutions. The concentrations of the peptide 2 antagonists are determined empirically (10 to 100 ng/ml). The incubation media are collected and analyzed for total androgen production. ³H-thymidine Ci/ml) is added to the cells for an additonal 4 hours incubation. The cells are then lysed and the cell lysates are filtered through DE81 ion-exchanger filters. The filter membranes are then put in vials filled with scintillation fluid and the radioactivity is measured by a Beckman β-counter.

In other experiments, the cells are cultured with different additions (e.g., diluted antibodies, pure SIP, synthetic peptide 2 or its antagonist, EGF, and/or other growth factors used as controls) for 72 hours and the cells are counted using a coulter counter. The viability of the cells is tested using MTT assay using a commercial kit. A similar experimental design is used to determine the effects of endogenous SIP on cell proliferation and steroidogenesis. A decrease in the number of proliferating cells or steroid production in the presence of antibodies and antagonists (in the absence of exogenous SIP) shows that SIP produced by these cells is required for their growth and steroid production

### In Vivo Experiments

SIP inhibitors (antibodies to peptide 2, and other peptide 2 antagonists) block the proliferation of cancer cells in vivo and result in reduced size of the tumor in athymic nude mice. Tumors develop readily in these mice due to a non-functional immune system. Ovarian (HEY, SKA) and prostate (DU145 and PC3) cancer cells (1 million cells) are injected under the skin of nude mice. The cells are mixed with the antibodies (1:1000) or antagonists (100 ng/ml) before injection. Alternatively, antibodies and antagonists are delivered at the site of injection at different time intervals (1, 5, 10 days) after the injection of cells. The production of tumor is monitored for 2 and 4 weeks by palpitation, and tumor sizes are recorded. At the end of 4 weeks, the animals are sacrificed, and the tumor is dissected for detailed analysis. An inhibition of tumor growth in mice receiving inhibitors shows that SIP produced by the tumor cells is required for tumor growth in vivo and that inhibition of SIP activity is useful for treatment of ovarian and prostate cancers. The doses of partially purified SIP is in the range of 10 to 50 microgram total protein/ml. The peptide 2 (SEQ ID NO:2) is used at the doses of 10 and 100 ng/ml. The antibody dilution is used at 1:1000.

### REFERENCES

1. Adams, L. Davey S., Scott K. 2002. The DING protein: an autocrine growth-stimulatory protein related to the human synovial protein. Biochimica et Biophysica Acta. 1586, 254-264.
2. Belenky M., Prasain J., Kim H., Barnes S. 2003. DING, a Genistein Target in Human Breast Cancer: A Protein Without a Gene. The Journal of Nutrition. Supp. 249S-2501S
3. Benton, L., Shan, L. X., Hardy, M. P. 1995. Differentiation of adult Leydig cells. J. Steroid Biochem. Mol. Biol. 53, 61-68.
4. Berna, A., Bernier F, Scott K, Stuhlmuller B. 2002. Ring up the curtain on DING proteins. FEBS Lett. 524 (1-3): 6-10.
5. Dickson, C., Webster, D. R., Johnson, H., Cecilia Millena, A., Khan, S. A. 2002. Transforming growth factor-beta effects on morphology of immature rat Leydig cells. Mol. Cell. Endocrinol. 195, 65-77.
6. Hager D A, Burgess R R 1980 Elution of proteins from sodium dodecyl sulfate-polyacrylamide gels, removal of sodium dodecyl sulfate, and renaturation of enzymatic activity: results with sigma subunit of Escherichia coli RNA polymerase, wheat germ DNA topoisomerase, and other enzymes. Annal Biochem 109:76-86
7. Ismail, A. A., Niswender, G. D., Midgley, A. R., Jr. 1972. Radioimmunoassay of testosterone without chromatography. J. Clin. Endocrinol. Metab. 34, 177-184.
8. Keck C, Khan S A, Rommerts F F G, Spiteri-Grech J, Sharma O P, Nieschlag E. (1993) The stimulation of rat Leydig cell steroidogenesis by human ovarian steroidogenesis inducing protein (SIP) does not require endogenous cAMP. Exp. Clin. Endocrinol. 101: 94-100.
9. Khan, S., Teerds, K., Dorrington, J. 1992a. Growth factor requirements for DNA synthesis by Leydig cells from the immature rat. Biol. Reprod. 46, 335-341.
10. Khan, S. A., Teerds, K., Dorrington, J. 1992b. Steroidogenesis-inducing protein promotes deoxyribonucleic acid synthesis in Leydig cells from immature rats. Endocrinology 130, 599-606.
11. Khan S A, Hallin P, Bartlett J, De Geyter Ch, Nieschlag E. (1988) Characterization of a factor from human ovarian follicular fluid which stimulates Leydig cell testosterone production Acta. Endocrinol 118: 283-293.
12. Khan S A, Keck Ch, Gudermann T, Nieschlag E. (1990) Isolation of a protein from human ovarian follicular fluid which exerts major stimulatory effects on in vitro steroid production of testicular, ovarian and adrenal cells. Endocrinology 126: 3043-3052.
13. Khan S A, Teerds K, Dorrington J H. (1992) Steroidogenesis inducing protein (SIP) promotes deoxyribonucleic acid synthesis in Leydig cells from immature rats. Endocrinology 130: 599-606.
14. Khan S A, Shum L W, Teerds K, Dorrington J H. (1992) Steroidogenesis inducing protein interacts with transforming growth factor-□ to stimulate DNA synthesis in rat granulosa cells. Mol. Cell. Endocrinol. 89: 97-103.
15. Khan S A, Donington J H, Moran M F. (1993) Steroidogenesis-inducing protein (SIP) stimulates protein-tyrosine kinase activity in rat Leydig cells. Endocrinology 132: 109-114.
16. Khan S A, Matysiak-Zablocki E, Ball R, Krtolica A, Hawkins G, Ghahremani M, Ludlow J W, Dorrington J. (1997) Steroidogenesis-inducing protein (SIP), isolated from human ovarian follicular fluid, is a potent mitogen for cell lines derived from ovarian surface epithelial carcinomas. Gynecologic Oncology 66: 501-508.
17. Krucher N A, Krtolica A, Lincoln J, Khan S A, Rodriguez-Rodriguez L, Ludlow J W. (1998) Mitogenic activity of steroidogenesis-inducing protein (SIP) during hypoxic stress of human ovarian carcinoma cells Cancer Letters 133: 205-214.
18. Manna, P. R., Kero, J., Tena-Sempere, M., Pakarinen, P., Stocco, D. M., Huhtaniemi, I. T. 2001. Assessment of mechanisms of thyroid hormone action in mouse Leydig cells: regulation of the steroidogenic acute regulatory protein, steroidogenesis, and luteinizing hormone receptor function. Endocrinology 142, 319-331.
19. Shan, L., Hardy, D. O., Catterall, J. F., Hardy, M. P. 1995. Effects of luteinizing hormone (LH) and androgen on steady state levels of messenger ribonucleic acid for LH receptors, androgen receptors, and steroidogenic enzymes in rat Leydig cell progenitors in vivo. Endocrinology. 136, 1686-1693.
20. Shan, L. X., Phillips, D. M., Bardin, C. W., Hardy, M. P. 1993. Differential regulation of steroidogenic enzymes during differentiation optimizes testosterone production by adult rat Leydig cells. Endocrinology. 133, 2277-2283.
21. Shultz, V. D., Phillips, S., Sar, M., Foster, P. M., Gaido, K. W. 2001. Altered gene profiles in fetal rat testes after in utero exposure to di(n-butyl)phthalate. Toxicol. Sci. 64, 233-242.
22. Stocco D M, Khan S A. (1992) Effects of steroidogenesis inducing protein (SIP) on steroid production in MA-10 mouse Leydig tumor cells: utilization of a non-cAMP second messenger pathway. Mol. Cell. Endocrinol. 84: 185-194.
23. Tena-Sempere, M., Barreiro, M. L., Gonzalez, L. C., Gaytan, F., Zhang, F. P., Caminos, J. E., Pinilla, L., Casanueva, F. F., Dieguez, C., Aguilar, E. 2002. Novel expression and functional role of ghrelin in rat testis. Endocrinology. 143, 717-725.
24. Watch, L., Morris, P. L. 2002. Cyclooxygenase 2 pathway mediates IL-1beta regulation of IL-1alpha, -1beta, and IL-6 mRNA levels in Leydig cell progenitors. Endocrinology. 143, 3276-3283.

TABLE 1
Amino Acid Substitutions

| Original Residue | Exemplary Substitutions |
|---|---|
| Arg | Lys |
| Asn | Gln |
| Asp | Glu |
| Cys | Ser |
| Gln | Asn |
| Glu | Asp |
| Gly | Pro |
| His | Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

TABLE 2

| Target Gene | Oligo-Primers | Size | Reference |
|---|---|---|---|
| SR-B1 | Sense 5′-CCA TTC ATG ACA CCC GAA TCC T-3′ | 100 bp | (Shultz |
| | (SEQ ID NO: 5) | | et al., 2001) |
| | Antisense 5′-TCG AAC ACC CTT GAT TCC TGG T-3′ | | |
| | (SEQ ID NO: 6) | | |
| StAR Sense | 5′-GCT CTG ATG ACA CCA CTC TGC-3′ | 276 bp | (Walch and |
| | (SEQ ID NO: 7) | | Morris, 2002) |
| | Antisense 5′-GTG GTA GAC CAG CCC ATG GA-3′ | | |
| | (SEQ ID NO: 8) | | |
| P450scc | Sense 5′-TTC CCA TGC TCA ACA TGC CTC-3′ | 100 bp | (Shultz |
| | (SEQ ID NO: 9) | | et al., 2001) |
| | Antisense 5′-ACT GAA AAT CAC ATC CCA GGC AG-3 | | |
| | (SEQ ID NO: 10) | | |
| 3□HSD | Sense 5′-CAG GAG CAG GAG GGT TTG T-3′ | 400 bp | (Tena-Sempere |
| | (SEQ ID NO: 11) | | et al., 2002) |
| | Antisense 5′-GTG GCC ATT CAG GAT | | |
| | (SEQ ID NO: 12) | | |
| P450c17 | Sense 5′-TGGCTTTCCTGGTGCACAATC | 100 bp | (Shultz |
| | (SEQ ID NO: 13) | | et al., 2001) |
| | Antisense 5′-TGAAAGTTGGTGTTCGGCTGAAG | | |
| | (SEQ ID NO: 14) | | |
| L19 | Sense 5′-GAA ATC GCC AAT GCC AAC TC-3′ | 405 bp | (Manna |
| | (SEQ ID NO: 15) | | et al., 2001) |
| | Antisense 5′-TCT TAG ACC TGC GAG CCT CA-3′ | | |
| | (SEQ ID NO: 16) | | |

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1.-37. (canceled)

38. A method of inhibiting the elaboration of steroidogenesis inducing protein (SIP) from a cell in a subject, comprising blocking the interaction of the cell with extracellular SIP, comprising contacting the extracellular SIP with an agent that binds to the extracellular SIP at a site of contact between the extracellular SIP and a cell membrane-bound receptor, thereby inhibiting interaction between the extracellular SIP with the receptor and inhibiting elaboration of SIP from the cell of the subject.

39. The method of claim 38, wherein the subject is a mammal.

40. The method of claim 39, wherein the mammal is human.

41. The method of claim 38, wherein the agent is an antibody that specifically binds the amino acid sequence identified as SEQ ID NO:2.

42. The method of claim 41, wherein the antibody is polyclonal.

43. The method of claim 41, wherein the antibody is monoclonal.

44. A method of inhibiting the proliferation of a cancer cell, comprising blocking the interaction of the cell with extracellular SIP, comprising contacting the extracellular SIP with an agent that binds to the extracellular SIP at a site of contact between the extracellular SIP and a cell membrane-bound receptor, thereby inhibiting interaction between the extracellular SIP with the receptor and inhibiting proliferation of the cell.

45. The method of claim 44, wherein the cancer is ovarian cancer.

46. The method of claim 44, wherein the cancer is testicular cancer.

47. The method of claim 44, wherein the cancer is prostate cancer.

48. The method of claim 44, wherein the cancer is breast cancer.

49. The method of claim 44, wherein the cancer is liver cancer.

50. The method of claim 44, wherein the cell is in vivo.

51. The method of claim 44, wherein the cell is in vitro.

52-56. (canceled)


SEQUENCE LISTING (16 sequences)

SEQ ID NO: 1 - protein, 8 aa, Artificial Sequence
  Description of Artificial Sequence; note = synthetic construct
EVQLVESG

SEQ ID NO: 2 - protein, 17 aa, Artificial Sequence
  Description of Artificial Sequence; note = synthetic construct
DVNGGGATLPQPLYQTA

SEQ ID NO: 3 - protein, 14 aa, Artificial Sequence
  Description of Artificial Sequence; note = synthetic construct
FNWYVDGVEVHNAK

SEQ ID NO: 4 - DNA, 51 bp, Artificial Sequence
  Description of Artificial Sequence; note = synthetic construct
gatgtgaacggcggcggcgcgaccctgccgcagccgctgtatcagaccgca

SEQ ID NO: 5 - DNA, 22 bp, Artificial Sequence
  Description of Artificial Sequence; note = synthetic construct
ccattcatgacacccgaatcct

SEQ ID NO: 6 - DNA, 22 bp, Artificial Sequence
  Description of Artificial Sequence; note = synthetic construct
tcgaacacccttgattcctggt

SEQ ID NO: 7 - DNA, 21 bp, Artificial Sequence
  Description of Artificial Sequence; note = synthetic construct
gctctgatgacaccactctgc

SEQ ID NO: 8 - DNA, 20 bp, Artificial Sequence
  Description of Artificial Sequence; note = synthetic construct
gtggtagaccagcccatgga

SEQ ID NO: 9 - DNA, 21 bp, Artificial Sequence
  Description of Artificial Sequence; note = synthetic construct
ttcccatgctcaacatgcctc

SEQ ID NO: 10 - DNA, 23 bp, Artificial Sequence
  Description of Artificial Sequence; note = synthetic construct
actgaaaatcacatcccaggcag

SEQ ID NO: 11 - DNA, 19 bp, Artificial Sequence
  Description of Artificial Sequence; note = synthetic construct
caggagcaggagggtttgt

SEQ ID NO: 12 - DNA, 15 bp, Artificial Sequence
  Description of Artificial Sequence; note = synthetic construct
gtggccattcaggat

SEQ ID NO: 13 - DNA, 21 bp, Artificial Sequence
  Description of Artificial Sequence; note = synthetic construct
tggctttcctggtgcacaatc

SEQ ID NO: 14 - DNA, 23 bp, Artificial Sequence
  Description of Artificial Sequence; note = synthetic construct
tgaaagttggtgttcggctgaag

SEQ ID NO: 15 - DNA, 20 bp, Artificial Sequence
  Description of Artificial Sequence; note = synthetic construct
gaaatcgccaatgccaactc

SEQ ID NO: 16 - DNA, 20 bp, Artificial Sequence
  Description of Artificial Sequence; note = synthetic construct
tcttagacctgcgagcctca